(19) United States
(12) Patent Application Publication
(10) Pub. No.: US 2025/0195153 A1
(43) Pub. Date: Jun. 19, 2025

(54) USER INTERFACE FOR SURGICAL ROBOTIC SYSTEM

(71) Applicant: PROCEPT BioRobotics Corporation, San Jose, CA (US)

(72) Inventors: Mark BAERENRODT, Woodside, CA (US); Yucheng ZHANG, San Jose, CA (US); Peter BENTLEY, Reno, NV (US); Angela ZHOU, Ramon, CA (US); Lewis CHEN, Mountain View, CA (US); Kee Sook JEON, Petaluma, CA (US); Lynne SMITH, St. Louis, MO (US)

(73) Assignee: PROCEPT BioRobotics Corporation, San Jose, CA (US)

(21) Appl. No.: 18/539,048

(22) Filed: Dec. 13, 2023

(51) Int. Cl. A61B 34/00; A61B 34/10; A61B 34/30; A61B 34/37; A61B 90/00

(52) U.S. Cl. CPC A61B 34/25; A61B 34/37; A61B 2034/107; A61B 2034/301; A61B 2090/3782

(57) ABSTRACT

A user interface for a surgical system is configured to proceed through a plurality of modes and display a current mode among the plurality of modes. Each of the plurality of modes is associated with one or more sub-modes, and the user interface is configured to proceed through the plurality of modes and the plurality of sub-modes in sequence. The user interface is configured to automatically generate an image from an image source for each of the modes and sub-modes and overlay one or more markers on the image along with instructions to the user. The user interface is configured to provide a control panel comprising plurality of frequently used user inputs below a centerline of a display for ergonomics, and to highlight a current mode of the sequence on a display relative to the other modes to show progression through the sequence of modes.

### RELATED APPLICATIONS

The subject matter of the present application is related to PCT/US2019/038574, filed Jun. 21, 2019, entitled “ARTIFICIAL INTELLIGENCE FOR ROBOTIC SURGERY”, published as WO/2019/246580 on Jun. 26, 2019, and U.S. application Ser. No. 18/163,187, filed on Feb. 1, 2023, “USER INTERFACE FOR THREE DIMENSIONAL IMAGING AND TREATMENT”, the entire disclosures of which are incorporated by reference.

### BACKGROUND

Prior methods and apparatus for treating patients can be less than ideal in at least some respects. Although surgical systems can be at least partially automated to perform at least some of the procedure in an automated manner and decrease user inputs, work in relation to the present disclosure suggests that the user interfaces of prior surgical systems can be less than ideal. For example, the prior user interfaces can be somewhat less intuitive than would be ideal. Although efforts to automate surgery have been successful, the set-up and treatment planning of a surgical system can rely on a user walking through several different screens of set-up and planning procedures. With these different screens, the user may have a less than ideal sense of the remaining steps to be completed and a general sense of how far along the user is in the process of setting up the system in relation to the total number of steps to be completed. Also, the user may configure different views from imaging devices more often than be ideal for different steps of the set up procedure, which can increase the set up time. At least some of the prior surgical systems can rely on dozens of user inputs to set up and plan a procedure, and the system controls may be less than ideally located, which may potentially result in less than ideal experience for the surgeon performing the surgery.

In light of the above, improved methods and apparatus are needed which would ameliorate at least some of the above mentioned aspects of prior surgical systems.

### SUMMARY

In some embodiments, a user interface of a surgical system is configured to proceed through a plurality of modes and display a current mode among the plurality of modes. In some embodiments, the plurality of modes comprises a sequence of modes, such as a set up mode, an align mode and a treatment mode. In some embodiments, each of the plurality of modes is associated with one or more sub-modes, and the user interface is configured to proceed through the plurality of modes and the plurality of sub-modes in sequence. In some embodiments, the user interface is configured to automatically generate an image from an image source for each of the modes and overlay one or more markers on the image along with instructions to the user, which can facilitate use of the system.

While the user interface can be configured in many ways, in some embodiments, instructions stored on a computer readable medium are configured to be executed by a processor to provide one or more features of the computer interface.

While the user interface can be configured in many ways, in some embodiments the user interface is configured to provide a control panel partitioned from an image display area, in which the control panel comprises a plurality of frequently used user inputs. The control panel portioned from the image display area can make it more intuitive for the user to move through modes of the procedure and visualize the images as the user advances the user interface through each of the modes.

In some embodiments, the user interface is configured to provide a plurality of frequently used user inputs below a centerline of a display, which can improve the ergonomics of the user interface. In some embodiments, the user interface is configured to provide a control panel below an image display area, in which the control panel comprises a plurality of frequently used user inputs. In some embodiments the user interface is configured to display one or more instructions to the user at an area of the control panel, which is where the user's attention is often directed.

In some embodiments, the user interface is organized into a plurality of modes and one or more corresponding sub-modes, which can help the user understand progress and streamline the procedure. In some embodiments, the current modes and sub-mode are displayed in a consistent area on the display, such as a fixed area of the display, which allows the user to understand progress by looking at the area while the system progress through the modes of operation. In some embodiments, the user interface is configured to provide a control panel, and an image display area at consistent locations on the display, which can make it easier for the user to understand where to look and where to provide inputs as the interface progresses through the modes and sub-modes.

In some embodiments, the user interface is configured to highlight a current mode of the sequence on a display, which makes it easier for the user to understand the relation of the current mode with other modes of the system in the sequence of modes. In some embodiments the user interface is configured to highlight the current mode and display one or more sub-modes associated with the current mode. In some embodiments, the one or more sub-modes associated with the current mode comprises a plurality of sub-modes and the current sub-mode is highlighted with respect to the other sub-modes associated with the current mode, which can help the user understand progress with respect to the current mode. In some embodiments, the highlighted mode and sub-mode are shown between forward and next icons, which can help the user associate the modes and sub-modes with the progression.

In some embodiments, the safety and ease of the procedure can be improved by automatically providing the ultrasound image as the primary image and the endoscope image as the secondary image during each of the modes subsequent to inserting the treatment probe, so that the surgeon can view and plan the treatment with the ultrasound image while the endoscope image is also available. In some embodiments, the processor instructions of the user interface are configured to select in sequence: 1) an ultrasound image source as the primary image source for the primary image display area in a first mode or sub-mode to insert an ultrasound probe into a patient; 2) an endoscope image source as the primary image source for the primary display area and the ultrasound image as the secondary image source for the secondary image display area in a second mode or sub-mode to insert a treatment probe comprising an endoscope into the patient; and 3) the ultrasound image source as the primary image source for the primary image display area and the endoscope image source as the secondary image source for the secondary image display area for each of a plurality of subsequent modes or sub-modes.

In some embodiments, the user interface is configured with an assisted planning mode, which allows the user to review and adjust a treatment plan generated with a tissue recognition algorithm, such as an artificial intelligence (AI) algorithm.

### INCORPORATION BY REFERENCE

All patents, applications, and publications referred to and identified herein are hereby incorporated by reference in their entirety, and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIGS. 1A and 1B show a surgical system, in accordance with some embodiments of the present disclosure;

FIG. 2 shows alignment of a user interface, a treatment probe and an imaging probe of the system of FIGS. 1A and 1B, in accordance with some embodiments;

FIGS. 3A to 3F show a user interface comprising display, in accordance with some embodiments;

FIG. 4A shows a treatment probe configured for use with a surgical system, in accordance with some embodiments;

FIG. 4B shows a motor-pack configured to couple to a treatment probe, in accordance with some embodiments;

FIG. 5 shows treatment probe and corresponding movements of an energy source, in accordance with some embodiments;

FIG. 6A shows an arm configured to support a treatment probe, in accordance with some embodiments;

FIG. 6B shows mounting device configured to couple to a motor-pack in an unlocked configuration, in accordance with some embodiments;

FIG. 6C shows the mounting device of FIG. 6B in a locked configuration, in accordance with some embodiments;

FIG. 7 shows an ultrasound imaging device, in accordance with some embodiments;

FIG. 8 shows an arm configured to support the ultrasound imaging device of FIG. 7, in accordance with some embodiments;

FIG. 9 shows a user interface method, in accordance with some embodiments; and

FIG. 10 shows a method of assisted tissue planning, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The presently disclosed systems and methods are well suited for use with many probes and diagnostic and surgical procedures. Although reference is made to a treatment probe comprising an energy source for prostate surgery and a transrectal ultrasound (“TRUS”) probe, the present disclosure is well suited for use with many types of probes inserted into many types of tissues, organs, cavities and lumens, such as brain, heart, lung, intestinal, eye, skin, kidney, liver, pancreas, stomach, uterus, ovaries, testicles, bladder, ear, nose, mouth, tumors, cancers, soft tissues such as bone marrow, adipose tissue, muscle, glandular and mucosal tissue, spinal and nerve tissue, cartilage, hard biological tissues such as teeth, bone, and lumens such as vascular lumens, nasal lumens and cavities, sinuses, colon, urethral lumens, gastric lumens, airways, esophageal lumens, trans esophageal, intestinal lumens, anal lumens, vaginal lumens, trans abdominal, abdominal cavities, throat, airways, lung passages, and surgery such as kidney surgery, ureter surgery, kidney stones, prostate surgery, tumor surgery, cancer surgery, brain surgery, heart surgery, eye surgery, conjunctival surgery, liver surgery, gall bladder surgery, bladder surgery, spinal surgery, orthopedic surgery, arthroscopic surgery, liposuction, colonoscopy, intubation, minimally invasive incisions, minimally invasive surgery, and others.

The presently disclosed systems and methods are well suited for combination with prior probes such as imaging probes and treatment probes. Examples of such probes include laser treatment probes, water jet probes, RF treatment probes, radiation therapy probes, ultrasound treatment probes, phaco emulsification probes, imaging probes, endoscopic probes, resectoscope probes, ultrasound imaging probes, A-scan ultrasound probes, B-scan ultrasound probes, 3D ultrasound probes, Doppler ultrasound probes, transrectal ultrasound probes, transvaginal ultrasound probes, longitudinal plane ultrasound imaging probes, sagittal plane ultrasound imaging probes, transverse plane ultrasound imaging probes, and transverse and longitudinal plane (e.g. sagittal plane) ultrasound imaging probes, for example.

The one or more images described herein can be generated in many ways, and may comprise one or more of a longitudinal image, a sagittal image, a parasagittal or a transverse image. In some embodiments, a longitudinal image comprises an image generated with an elongate imaging probe, in which the longitudinal image extends along a plane corresponding to an elongate axis of the imaging probe, and the transverse images extend along a plane that is transverse to the elongate axis of the probe, for example substantially perpendicular to the elongate axis of the probe and the corresponding longitudinal images. The elongate probe can be inserted into the patient with any suitable orientation. In some embodiments, the probe is inserted into the patient substantially along a midline of the patient, such that the longitudinal images correspond to sagittal images of the patient. In some embodiments, the elongate imaging probe comprises a TRUS probe, and the longitudinal images comprise sagittal images, although other probes with different orientations can be used to generate images in accordance with the present disclosure. Although reference is made to ultrasound probes inserted into the patient, in some embodiments the imaging device comprises an external imaging probe in which the longitudinal and transverse images can be referenced to one or more arrays of the external imaging probe. In some embodiments, the one or more images are generated from a 3D tomographic image data set such as a Digital Imaging and Communications in Medicine (DICOM) image data set.

The presently disclosed systems, methods and apparatuses are well suited for combination with many prior surgical procedures, such as water jet enucleation of the prostate, transurethral resection of the prostate (TURP), holmium laser enucleation of the prostate (HOLEP), prostate brachytherapy, and with surgical robotics systems and automated surgical procedures. The following patent applications describe examples of systems, methods, probes and procedures suitable for incorporation in accordance with the present disclosure: PCT/US2013/028441, filed Feb. 28, 2013, entitled “AUTOMATED IMAGE-GUIDED TISSUE RESECTION AND TREATMENT, published as WO 2013/130895; PCT/US2014/054412, filed Sep. 5, 2014, entitled “AUTOMATED IMAGE-GUIDED TISSUE RESECTION AND TREATMENT”, published as WO 2015/035249; PCT/US2015/048695, filed Sep. 5, 2015, entitled “PHYSICIAN CONTROLLED TISSUE RESECTION INTEGRATED WITH TREATMENT MAPPING OF TARGET ORGAN IMAGES”, published as WO2016037137; PCT/US2019/038574, filed Jun. 21, 2019, entitled “ARTIFICIAL INTELLIGENCE FOR ROBOTIC SURGERY”, published as WO2019246580A1 on Dec. 26, 2019; PCT/US2020/021756, filed Mar. 9, 2020, entitled “ROBOTIC ARMS AND METHODS FOR TISSUE RESECTION AND IMAGING”, published as WO/2020/181290; PCT/US2020/058884, filed on Nov. 4, 2020, entitled “SURGICAL PROBES FOR TISSUE RESECTION WITH ROBOTIC ARMS”, published as WO/2021/096741; PCT/US2021/070760, filed on Jun. 23, 2021, entitled “INTEGRATION OF ROBOTIC ARMS WITH SURGICAL PROBES”, published as WO/2021/263276; and PCT/US2021/038175, filed on Jun. 21, 2021, entitled “SYSTEMS AND METHODS FOR DEFINING AND MODIFYING RANGE OF MOTION OF PROBE USED IN PATIENT TREATMENT”, published as WO/2021/262565; the entire disclosures of which are incorporated herein by reference.

In some embodiments, an energy source is used to treat tissue. The energy source may comprise any suitable energy source, such as one or more of an electrode, a loop electrode, a laser source, a thermal energy source, a mechanical energy source, a mechanical sheer, an ultrasound probe, a cavitating ultrasound probe, a water jet, e.g. a fixed pressure water jet, a plasma source, a steam source, a morcellator, a trans urethral needle, a photo ablation source, a radiation energy source, a microwave energy source or a water jet evacuation source. The energy source can be combined with other treatments and compounds, such as photochemical treatment agents. The imaging probe may comprise any suitable probe, such as endoscopic probes, resectoscope probes, ultrasound imaging probes, A-scan ultrasound probes, B-scan ultrasound probes, Doppler ultrasound probes, transrectal ultrasound probes, transvaginal ultrasound probes, sagittal plane ultrasound imaging probes, transverse plane ultrasound imaging probes, and transverse and sagittal plane ultrasound imaging probes, for example.

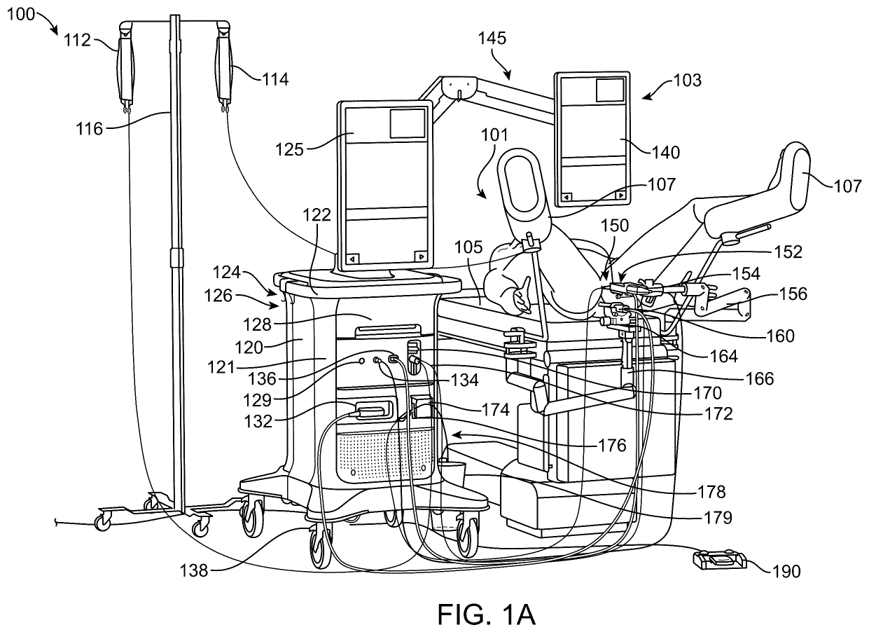

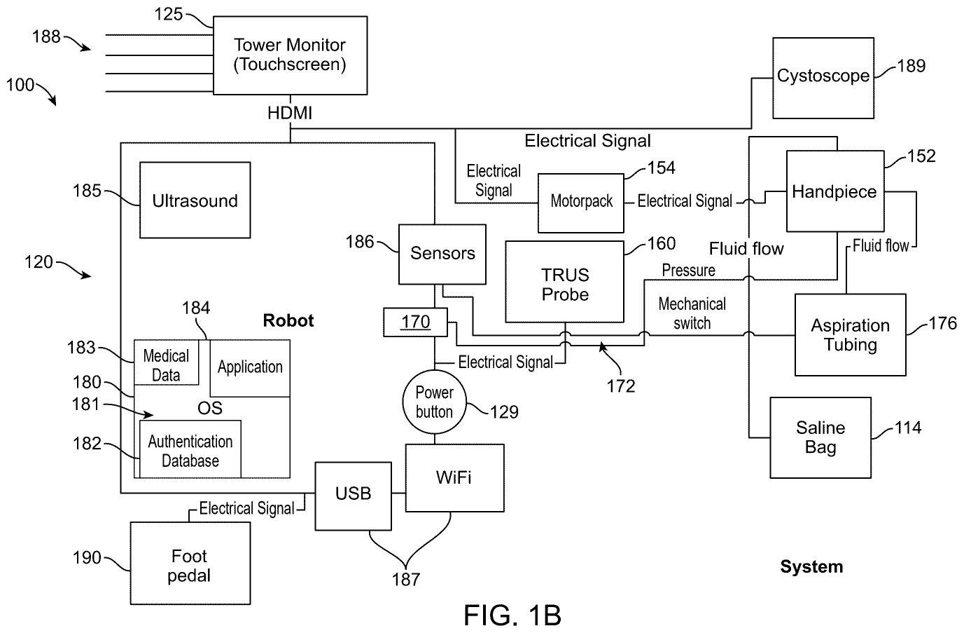

FIG. 1A shows a surgical system 100, in accordance with some embodiments. The system 100 comprises a console 120 which comprises several components of the system 100, such as electronics, circuitry and one or more processors to control a treatment probe 150, which has been inserted into the patient 101. The system 100 comprises a user interface 103 comprising a monitor, such as a touch screen display 140. The display 140 is coupled to an adjustable arm 145, which allows the display to be positioned above a midline of patient near an insertion location of the probe 150, which can allow the user such as a surgeon to control the surgery when seated in front of the display 140 and near the surgical instruments, such as the treatment probe 150 and an imaging probe 160.

The console 120 can be configured and sized and shaped in many ways. In some embodiments, the console 120 comprises a tower, in which the horizonal dimensions of the console are less than a vertical height of the console. Alternatively or in combination, the console 120 may comprise a cart, for example with lockable wheels, which allow the console to be moved.

The user interface 103 can be configured in many ways and may comprise a display, such as a touch screen display, for example. Although reference is made to a touch screen display, the user interface 103 positioned above the patient may comprise one or more components that are not shown on the display, such as a positioning pad, one or more input buttons, a pointing device such as a track pad, a knob, or other suitable component. These components of the user interface 103 can be located to the side of the display 140, for example. Alternatively or in combination, the display may comprise one or more input features shown on the touch screen display touch screen display, such as buttons, a positioning pad, and features configured for the user to swipe the display to provide user input.

In some embodiments, the system 100 comprises a patient support 101. The patient support may comprise any suitable support, such as a bed, for example. The support 101 can be configured to support the patient such that a midline of the patient is approximately aligned with a midline of the support 101. The support 105 may comprise one or more stirrups 107, which allow the legs and feet of the patient to be elevated during surgery. In some embodiments, the support 101 is configured to place the patient in a lithotomy position, for example. The support may comprise adjustable features such as one or more motors to raise and lower the patient for example.

In some embodiments, the system 100 comprises one or more probes inserted into the patient. The one or more probes may comprise a treatment probe 150. While the treatment probe 150 can be configured in many ways, in some embodiments the treatment probe 150 comprises an energy source configured to deliver energy to the patient to treat tissue as described herein. In some embodiments, the treatment probe is configured to be inserted into an external opening of the urethra of the patient, although other access locations can be used, such as surgical opening to introduce the probe. In some embodiments, the one or more probe inserted into the patient comprises an imaging probe, such as an ultrasound probe, for example. In some embodiments, the imaging probe inserted into the patient comprises a transrectal ultrasound (TRUS) probe 160. Although reference is made to a separate ultrasound probe, in some embodiments the ultrasound imaging transducer array is inserted into the patient through the same opening as the treatment probe and may located on the same probe as the energy source. Also, the ultrasound imaging device may comprise an external ultrasound imaging array, for example.

The one or more probes inserted into the patient may be coupled to one or more arms to support the one or more probes. The one or more arms may comprise a treatment probe arm 156 configured to support the treatment probe 150 during treatment, for example with the treatment probe 150 inserted into the patient. Alternatively or in combination, the one or more arms may comprise an arm to support the imaging probe, such as an arm 166 configured to support a TRUS imaging probe. In some embodiments, the TRUS probe is coupled to the arm 166 with a stepper 164, which allows the probe 166 to be advanced along the rectum or the colon of the patient and to be drawn proximally toward the surgeon with rotation of a knob with the TRUS probe 160 inserted into the patient 101. In some embodiments, the stepper 164 comprises a cradle for rotationally adjusting the TRUS probe 160 to align with patient anatomy.

The treatment probe 150 can be configured in many ways, and may comprise a handpiece 152 configured to allow the probe to be inserted into the patient. The handpiece 152 can be configured to couple to a source of mechanical energy, such as a motor-pack 154, which is configured to drive one or more components of the treatment probe. In some embodiments, the treatment probe is configured to receive an endoscope such as a cystoscope in order to allow visualization of the treatment area and to facilitate insertion of the treatment probe. In some embodiments, the endoscope is incorporated into the probe as an integral part of the probe.

The console 120 can be configured in many ways, and may comprise internal electronics, processors, circuitry and connectors configured to couple to other components of the surgical system. In some embodiments, the console 120 comprises one or more handles 122 and wheels such as locking wheel 138, which allow the console to be moved by up to two people at a time, for example to be stored or brought into an operating room for surgery and locked into position. The console 120 may comprise additional features to facilitate surgery and use of the system, such one or more of a motor-pack holder 124, a TRUS probe holder 126, a drawer 128, or a power button 129, for example. In some embodiments, the motor-pack holder 124 and the TRUS probe holder can located on either side of the console 120, depending on user needs. The console 120 may comprise connectors to couple to one or more components of the surgical system 100, such as one or more of a TRUS probe connection 132 configured to couple the console to the TRUS probe 160, a motor-pack connection 134 configured to couple to the motor-pack 154, or a scope connection 136 configured to couple to an endoscope such as a cystoscope.

In some embodiments, the system 100 comprises a water jet pump cartridge 170, which is received in a receptacle of console 120. In some embodiments, a water jet line 172 extends from the pump cartridge 170 to the treatment probe 150 to provide a water jet from the treatment probe. In some embodiments, the console 120 comprises an aspiration pump 174, which is coupled to probe 150 with an aspiration line 174.

In some embodiments, the system 100 comprises a waste line 178 configured to couple to a waste container 179. The waste line 178 can be configured to receive fluids and other material from the patient via the treatment probe 150, for example. In some embodiments the waste line 178 is coupled to the aspiration pump 176, although line 178 may receive fluids from other sources, for example.

In some embodiments, the system 100 comprises a foot pedal 190 configured for a user such as a physician to control treatment, such as starting, pausing, resuming or stopping the treatment for example.

In some embodiments, the console 120 comprises a front side 121 in which a plurality of connectors are plugged into the console 120, which allows the user such as a surgeon to readily view the connectors to ensure that these and the associated lines have been properly connected. The plurality of connectors located on the front side 121 may comprise one or more of the TRUS probe connection 132, the motor-pack connection 134, the scope connection 136, the water jet pump cartridge 170, the water jet line 172, the aspiration pump 174, the waste line 178, or the connector of the foot pedal 190. This location of the connectors and lines allows a user such as a surgeon to readily view the connectors to ensure that the appropriate connections have been made, and may help diagnose system 100 if a fault is detected, for example.

In some embodiments, system 100 comprises a source of fluids, such as a liquid, e.g. saline, although other fluids such as gasses for insufflation may be provided. In some embodiments, a saline source 112 is coupled to water jet pump cartridge 170. In some embodiments, a saline source 114 is coupled to treatment probe 150 in order to flush the surgical site with a fluid such as saline, for example. In some embodiments, one or more of the saline sources is supported with a pole 116 to provide saline to one or more of the pump or the probe 150 with gravity assisted flow, for example. The pole 116 may comprises a pole that is separate from the console 120 or a pole that is integrated with the console 120, for example so as to extend upwardly from the console.

In some embodiments, the system 100 comprises a second display 125, which is supported with the console 120. The second display 125 can be helpful for staff to view the surgery and to assist the surgeon. In some embodiments, the second display 125 mirrors display 140. The second display 125 may have similar user input features as the display 140, or decreased functionality to ensure the physician retains control of the treatment.

In some embodiments, the screen of the second display 125 comprises annotation input features for staff or a proctor to provide annotation inputs for the surgeon to view on the first display 140, for example without controlling system functions from the second display.

FIG. 1B shows a schematic diagram of some components of system 100. In some embodiments, the console 120 comprises one or more processors 180. The one or more processors 180 can be configured with instructions to perform various functions of system 100. In some embodiments, the one or more processors 180 comprise an operating system 181, an authentication database 182 to provide user authentication, a medical database to store medical data of patients, an application block 184 to run one or more applications of the system, such as treatment planning and user interface (UI) processes, instructions to the motors to move the linkage of treatment probe 150, and system monitoring functions as describe herein. In some embodiments, the console 120 comprises imaging circuitry such as ultrasound circuitry 185 housed within the console 120. In some embodiments, the one or more processors 180 is configured to give different users different levels of access 188 such as full system access or partial access to system 100. The access 188 can be provided through a console user interface, for example via the console display 125, which may comprise a touch screen display, for example. In some embodiments, the system access is provided to administrators, such as a urology administrator of the local health care provider, hospital information technology administrators, and representatives of the manufacturer of system 100, such as field service representatives. In some embodiments, the system 100 comprises communication circuitry 187 housed within console 120, such as universal serial bus (USB) circuitry and wireless fidelity (WiFi) circuitry, for example.

While the console 120 can be configured in many ways, in some embodiments, the console 120 comprises internal components, such as an internal computer system and an internal ultrasound imaging system housed within console 120. In some embodiments, the console 120 comprises a tower configuration, in which the console comprises a height greater than a width of the console.

The console 120 can be coupled to the components of system 100 with one or more connectors such as electrical connectors or fluidic connectors as described herein. In some embodiments, the console 120 is coupled to the console display 125 with an electrical connector such as an HDMI cable. The console can be connected to the endoscope such as cystoscope 189 with an electrical line to transmit electrical signals, in order to display images on the displays. The motor-pack 124 can be coupled to the console 120 with one or more electrical lines such as cables, to transmit movement signals and provide feedback to the one or more processors within console 120. The motor-pack 154 can be coupled to the handpiece 152 with one or more electrical lines to transmit electrical signals between the handpiece and the motor-pack 154 and the one or more processors 180, such as encoder signals and feedback signal related to movement of the energy source on the probe provided by a linkage housed within the handpiece 152. The one or more sensors 186 can be coupled to the one or more processors 180 with internal circuitry housed within console 120 to monitor fluid flow, for example. The one or more sensors 186 can be coupled to the aspiration pump, for example. In some embodiments, the one or more sensors 186 is coupled to the handpiece 152 with aspiration tubing 176 to monitor the removal of material from handpiece 152. In some embodiments, the saline source 114 such as the saline bag is coupled to the handpiece 152.

In some embodiments, the ultrasound imaging device such as TRUS probe 160 is coupled to the console 120 with one or more electrical lines, such as an electrical cable to couple the ultrasound imaging device such as TRUS probe 150 to the ultrasound circuitry 185.

In some embodiments, the console 120 comprises one or more sensors 186 coupled to the aspiration line 176 to measure fluid flow from the handpiece 152.

In some embodiments, the system 100 comprises an endoscope such as cystoscope 189, configured to view optical images of tissue at the surgical site. The endoscope such as cystoscope 189 may be coupled to the handpiece 152 and the probe 150 as described herein.

In some embodiments, a pump cartridge 170 is insertable into a receptacle of the console, in order to provide pressurized water to the energy source such as a waterjet on the treatment probe. In some embodiments, the high pressure line 172 couples the pump cartridge to the water jet on the handpiece.

In some embodiments, the foot pedal 190 is coupled to the console 120 with one or more electrical lines to transmit signals, such as a cable, in order to control system 100 as described herein.

The arm 145 can be configured in any way to support the surgeon display 140 above the patient as described herein. In some embodiments, the arm 145 comprises a plurality of joints and one or more of a locking arm, a clutch arm, a manual arm, a gas-spring arm, a robotic arm, a zero-gravity arm or a zero gravity robotic arm, for example. In some embodiments, the arm 145 comprises a gas spring arm with a gas strut and a spring.

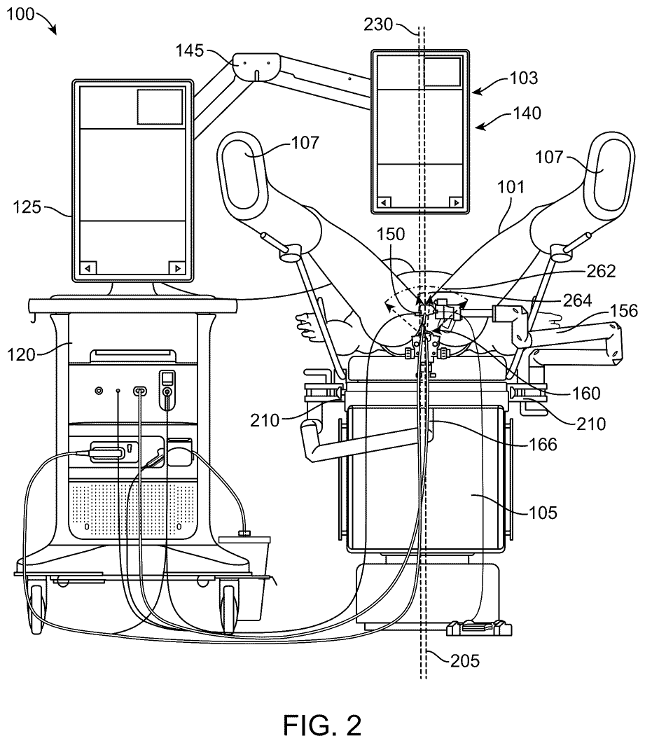

FIG. 2 shows alignment of a user interface 103, a treatment probe 150 and an imaging probe 160 of the system 100, in accordance with some embodiments. In some embodiments, the patient comprises a midline 205, and the user interface 103 and the treatment probe 150 are arranged with respect to the midline of the patient. In some embodiments, the monitor is poisoned such that a plane corresponding to the midline of the patient extends through the monitor, which can help with visualization of the images shown on the display. This location is also helpful because it allows the surgeon to readily reach the input controls of the user interface 103, which comprises the display 140. Readily reaching the input controls can be helpful to decrease fatigue associated with controlling the surgical system 100, for example when several surgeries are performed in a day or with long surgeries lasting more than an hour, for example.

In some embodiments, the arm 145 is configured to support the user interface 103 comprising the surgeon display 140 coupled to the positioning arm, in which the positioning arm 145 is configured to support the display at a first location above a midline of the patient with a first configuration. In some embodiments, the arm 145 is configured to place the surgery display 140 at a second location away from the midline of the patient with a second configuration for storage as described herein.

While the arm 145 can be configured in many ways, in some embodiments the arm is configured to allow movement of the surgeon display 140 for visualization and interaction by the surgeon while surgeon may be on one knee for insertion of the ultrasound probe such as TRUS probe 160, for example. In some embodiments, the display is configured to receive user inputs when covered with a sterile drape, and the inputs are provided through the sterile drape, and the sterile drape may comprise a substantially transparent drape to allow the user to see the display through the drape and provide inputs through the drape. The arm 145 can be configured for the surgeon to move the display 140 out of the way during handpiece insertion, for example by pushing on display 140 to rotate the arm 145 relative to the console 120 to position the display away from the surgeon and superiorly above the patient for example while using sterile technique with display at least partially draped and the surgeon wearing surgical gloves. The display 140 can then pulled in toward the physician while seated or standing for touch-screen intensive steps after insertion and alignment of probes as described herein.

In some embodiments, the imaging probe such as TRUS probe 150 is arranged with respect to the midline of the patient, the treatment probe 150 and the display 140. In some embodiments, this arrangement can assist the physician with understanding the physical locations of the probes in front of them and coordinating movements of the probes in relation to internal images of the patient shown on the display. For example, the physician can move a proximal end of a probe in order to manually move a distal portion of the probe, e.g. manually translate or rotate the probe, or use instrument controls from the user interface 103 in order to move one or more of the probes to a desired location within the patient, such as with translation or rotation of the probe.

In some embodiments, the imaging device such as TRUS probe 160 is configured to generate longitudinal images such as sagittal images and transverse images. In some embodiments, the longitudinal images correspond to an imaging plane along with a longitudinal image of the probe, and the transverse images correspond to images along plane transverse to the elongate axis of the imaging probe. In some embodiments, the transverse images correspond to a transverse field of view 262, which corresponds to a transverse image plane. In some embodiments, the transverse field of view corresponds to an angle of rotation about an elongate axis of the imaging probe such as TRUS probe 260. With the monitor placed over the midline of the patient, the physician can more readily associate the transverse images shown on the display with the physical locations of the corresponding tissue that is shown in the images.

In some embodiments, the imaging device such as TRUS probe 260 is configured to generate longitudinal images, e.g. sagittal images, along a longitudinal image plane within a longitudinal image field of view 624, which may comprise a sagittal image along a sagittal image field of view. In some embodiments, the monitor is aligned with the patient at a location above the sagittal image plane, such that the plane of the sagittal image extends through the monitor, for example. This alignment of the display with the longitudinal image plane allows the physician to associate the position of the probes more readily with the longitudinal images. Alternatively or in combination, the monitor can be located inferiorly or superiorly, with respect to the patient, such that the plane of the transverse image is within about 10 inches of the image display area of the monitor. In some embodiments, this positioning of the monitor can improve the physician's ability to associate the transverse and sagittal images shown on the display with the physical locations of the probes and corresponding tissue that is shown in the images.

In some embodiments, the transverse image shown on the display approximately parallel to the transverse image plane. In some embodiments, the transverse image plane is parallel to the image shown on the display to within 30 degrees, or within 10 degrees, for example. In some embodiments, the ultrasound imaging device comprises an ultrasound array configured to generate a longitudinal image along a longitudinal image plane, the arm configured to support the display with longitudinal image displayed on the display above the patient.

In some embodiments, the longitudinal image plane corresponds to a plane extending from the ultrasound transducer array and through a portion of the display. In some embodiments, the transducer device is rotatable about a longitudinal axis in order to rotate an angle of the longitudinal image plane to view the treatment probe with the plane extending through the transducer array, the treatment probe and the monitor.

In some embodiments, the treatment probe extends through at least a portion of the longitudinal image plane. In some embodiments, the longitudinal image shown on the display is approximately perpendicular to the longitudinal image plane and optionally to within 30 degrees of perpendicular.

In some embodiments, the ultrasound imaging device such as TRUS probe 160 comprises a transverse array and a longitudinal array as described herein, and the arm 145 is configured to position the image shown on the display to within 30 degrees of parallel to a long axis of the longitudinal array and to within 30 degrees of perpendicular to a long axis of the transverse array.

In some embodiments, patient support 105 is configured to support the patient with the patient support below patient, the midline 205 of the patient corresponding to a plane extending through the display and the support.

In some embodiments, the stirrups 107 are configured to support the legs of the patient splayed and the feet of the patient above a torso of the patient, and wherein the surgeon display 140 is sized to fit between the legs of the patient.

In some embodiments, the arm 145 is configured to support the surgeon display 140 above the patient and superiorly relative to a penile fenestration of a drape covering the patient, which the treatment probe 150 enters the patient.

While the surgeon user interface 103 and display 140 can be configured in many ways, in some embodiments, display 140 has a height and a width, wherein the height is greater than the width. In some embodiments, the positioning arm 145 is configured to align a vertical centerline 230 of the display 140 with a midline of the patient 205 and a corresponding midline of the patient support. In some embodiments, the positioning arm 145 is configured to place a vertical centerline 230 of the display within about 2 inches of a midline 205 of the patient with the patient on the support 105.

In some embodiments, the positioning arm 145 is configured to place a centerline of the touchscreen display 140 to within 2 inches of a centerline of the treatment probe, for example a longitudinal axis of probe 150.

In some embodiments, the surgeon display 140 comprises a touch screen display configured to receive a plurality of user inputs through a drape placed over the touch screen display to provide sterility. The arm 145 is configured to resist movement in the extended configuration in response to the user input through the drape. In some embodiments, the touchscreen display 140 is configured to receive a plurality of user inputs from a finger of a user covered with a glove transmitted from the finger through the glove and the drape to the touchscreen display, and the arm is configured to resist movement in response to the user input transmitted through the glove and the drape. In some embodiments, the drape comprises a sterile drape and the glove comprises a sterile glove.

While the arm 145 can be configured in many ways, in some embodiments the arm comprises a locking arm configured to lock the arm in place in the extended configuration above the patient. In some embodiments, movement of the display is inhibited with a locking mechanism, such as a turn screw, lever or clamp, for example.

Alternatively or in combination, the arm can be configured not to move substantially when subjected to forces associated with inputs to display 140, such as touch inputs, and to move in response to greater amounts of force to reposition the display 140 supported with arm 145. In some embodiments, the positioning arm is configured to resist movement of the touchscreen display when placed above the midline 205 and touched by a user with a first amount of force to input data and to allow movement of the display with a second mount of force greater than the first amount of force. In some embodiment, the first amount force is within a range from about 0 (e.g. 0.01 or 0.1) pounds to about 5 pounds and optionally within a range from about 0 (e.g. 0.01 or 0.1) pounds to about 2 pounds. In some embodiments, the second amount of force is greater than about 2 pounds and optionally greater than about 5 pounds. The arm can be configured with stiction to inhibit movement of the display when the first amount of force is applied touch screen when the touch screen is positioned above the midline of the patient and to allow movement with the second amount of force greater than the first amount of force, for example. The arm 145 may comprise frictional pads configured to resist movement with forces below the threshold amount of force and to allow movement with forces above the threshold amount.

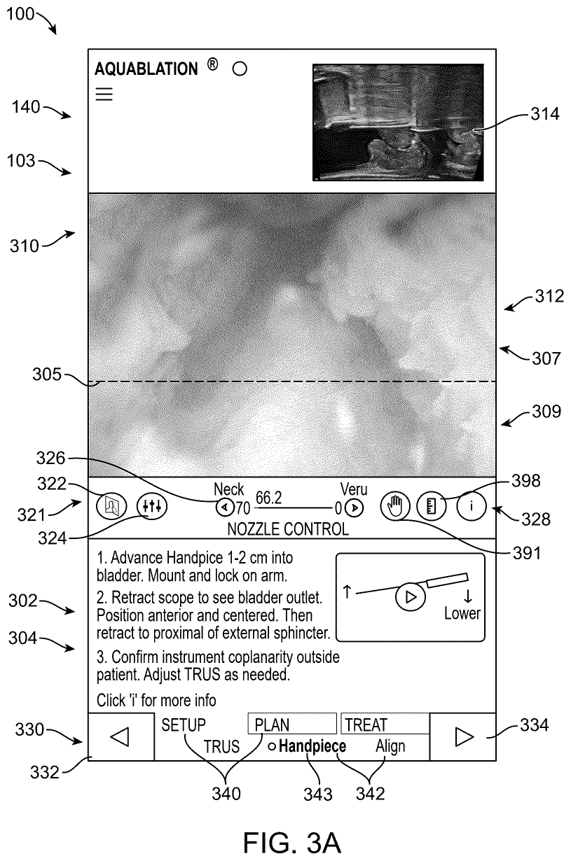

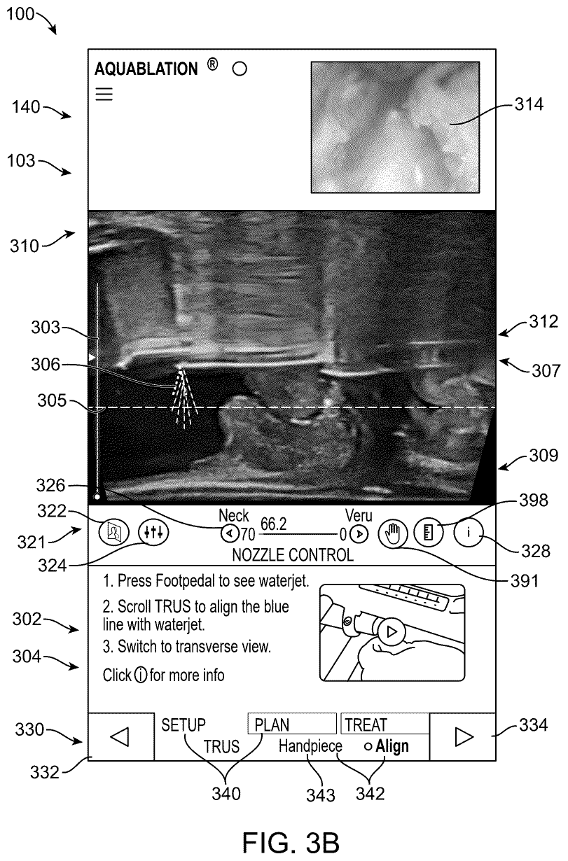

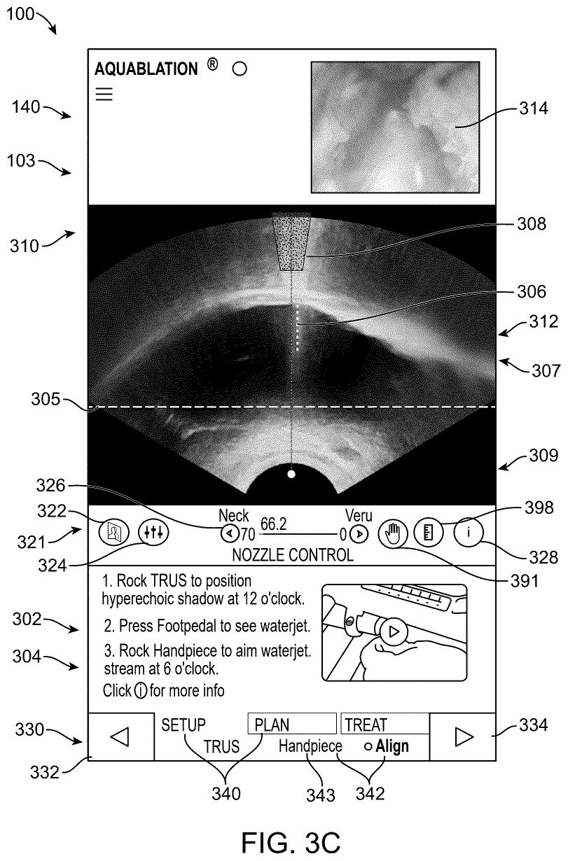

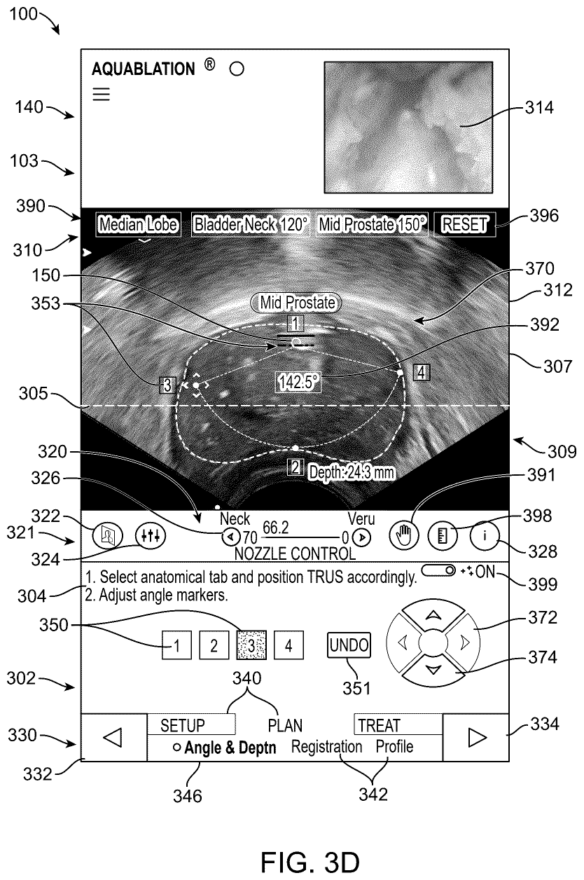

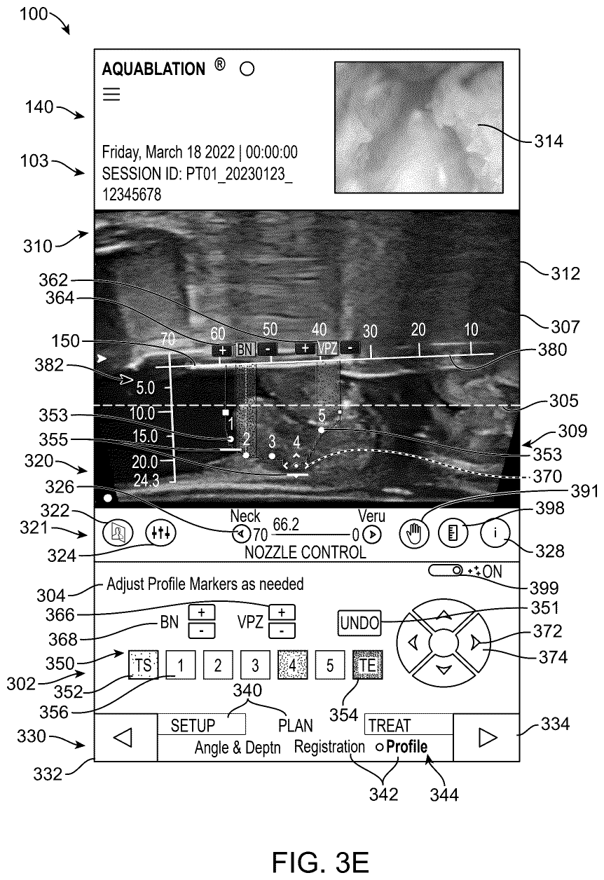

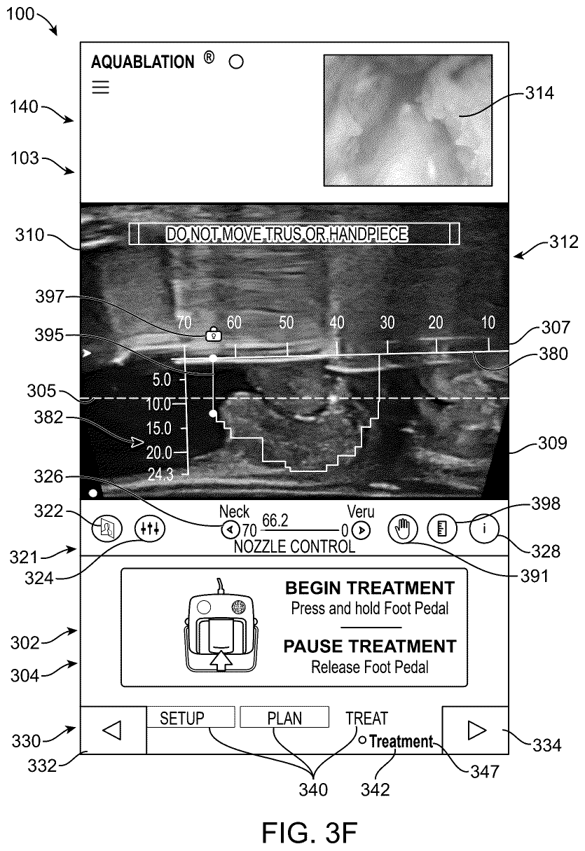

FIGS. 3A to 3F show a user interface 103 comprising a display 140 of the system 100, in accordance with some embodiments. In some embodiments, the display 140 comprises a portrait orientation to improve placement of the monitor above the patient as described herein. In some embodiments, the user interface 103 comprises an image display area 310 and a control panel 302, in which the control panel 302 is partitioned from the image display area. In some embodiments, the control panel 302 is located on an area of the display that is separate from the image display area 310, such that the control panel and image display area appear on different areas of the display. In some embodiments, the control panel 302 is located below the image display area 310. Alternatively, the control panel can be located above the image display area or alongside the image display area, for example with the display in a landscape configuration.

Although reference is made to a control panel 302 in the context of a touch screen display, one of ordinary skill in art will recognize that the control panel 302 may comprise one or more hardware components instead of a touchscreen area of the display 140 as described herein. The control panel 302 located below the image control area can facilitate user control and decrease the likelihood that the user will reach as far upward to provide inputs to user interface 103 as compared to the image control panel being located above the image display area.

In some embodiments, the user interface 103 is configured for the user to touch the user interface such as the touchscreen display a total number of times at a plurality of locations to set up the treatment, plan the treatment and perform the treatment, and at least half of the plurality of locations touched the total number of times is located below a centerline of the display, which can improve ergonomics and facilitate the user providing input to the user interface. In some embodiments, at 75% of the plurality of locations touched the total number of times is located below a centerline of the display. In some embodiments, at 90% of the plurality of locations touched the total number of times is located below a centerline of the display. In some embodiments, the total number of times the user touches the user interface at the plurality of locations to set up the treatment, plan the treatment and perform the treatment comprises a total number of touches at least 50 times, a total number of touches of at least 100 times, a total number of touches of at least 150 times, a total number of touches of at least 200 times, or a total of number of touches of at least 250 times.

In some embodiments an upper area 307 of the display is located above a horizontal centerline 305 and a lower area 309 of the display is located below the horizontal centerline 305. In some embodiments, at half of the image display area 310 is located above the centerline and at least half of the area of the control panel 302 is located below the centerline 305, which can improve ergonomics because the control panel can be more readily reached by the user as compared to the control panel located above the image display area. In some embodiments the user will touch the control panel at least twice as often as the image display area, and in some embodiments the user will touch the control panel at least four times as often as the image display area. In some embodiments, at least 75% of the image display area 310 is located above the horizontal centerline and at least 75% of the control panel is located above the image display area. In some embodiments, at least 90% of the image display area 310 is located above the horizontal centerline and at least 90% of the control panel is located above the image display area. In some embodiments, the entire image display area 310 is located above the horizontal centerline and the entire control panel is located above the image display area.

The image display area 310 can be configured in many ways and may comprise a primary image area 312 and a secondary image area 314. The secondary image display area can be located outside the primary image display area. Alternatively or in combination, the secondary image may be shown within a portion of the primary image, for example with a picture in picture configuration. In some embodiments, the primary image display area is larger than the secondary image display area.

The control panel 302 can be configured in many ways, and may comprise a tool bar 321, for example. The tool bar 302 may comprise one or more of a plane toggle icon 302 for a user to toggle between sources of the primary image and the secondary image in response to a user input to the icon, an image setting icon 324 for a user to select to adjust image settings, a longitudinal control icon 326 for a user to adjust a longitudinal position of an energy source on the treatment probe, a measurement icon 398 for a user to select to measure distances between structures shown in an image, or an information icon 328 for a user to select to display information such as information about the surgical system and the patient, for example. Although reference is made to plane toggle icon 302, image setting icon 304, longitudinal control icon 326, measurement icon 398 and information icon 328 in the context of tool bar 321, one or more of these icons and controls can be located elsewhere on the display, for example.

The icons shown on the display may comprise any suitable symbol to indicate the functionality of the icon. In some embodiments, the icon is configured for a select the icon with a user input such as touching the display at a location corresponding to the icon. In some embodiments, the plane toggle icon 322 is configured to receive a user input such as the user touching the icon to toggle the primary image and the secondary image source.

The settings icon such as the image setting icon 324 can be configured in many ways, and may comprises any suitable icon such as a gear, slider or other icon that conveys to the user that settings can be adjusted with the icon. In some embodiments, the image setting icon 324 shows a plurality of sliders to indicate to the user that the user can adjust the image settings. In some embodiments, the user touching an icon to provide user input results in a pop up window, such as a pop up window for the user to adjust the imaging settings, which may comprise imaging setting of one or more of the endoscope or the TRUS probe. In some embodiments, the icon may comprise a plurality of controls associated with the icon. For example, the longitudinal control icon 326 may comprise a first icon such as an arrow pointing in a first direction to indicate to the user that an energy source is moved in the first direction in an image shown on the display, e.g. to advance a probe carrying the energy source, and a second icon such as an arrow pointing in a second direction to indicate to the user that the energy source is moved in the second direction in an image shown on the display, e.g. to retract the probe carrying the energy source. The longitudinal control 326 may comprise a third icon, such as a bar, to show the position of the energy source on the probe, for example.

In some embodiments, the user interface is configured to work with a first display 140 such as a surgeon display and a second display 125 for staff and other people associated with the surgical procedure. In some embodiments, a display control icon 391 allows a user to adjust input settings of the first display 140 and the second display 125 to allow limited inputs with the second display 125 while the surgeon controls the inputs with surgeon display 140. In some embodiments, the display control icon 391 comprises an annotation enable toggle that allows annotations to be input from the second display 125 when enabled and does not allow annotations to be input from the second display 125 when disabled. In some embodiments, the display control icon 391 is configured to provide a pop-up window that allows the user of the first display 140 to allow at least some user input from the second display 140, such as annotations. In some embodiments, the first display 140 comprises a touch screen display configured for a user such as a surgeon to control and plan the surgery with inputs as described herein, and the second display 125 is configured to substantially mirror the first display 140, which allows the operating room staff to readily view the surgical procedure on the second display 125 in synchrony with the surgeon viewing the first display 140.

In some embodiments, only the first display 140 is configured to accept and confirm the treatment plan from the surgeon touchscreen display 140, even when annotations at the second display are enabled. In some embodiments, the second display 125 is configured to allow limited user inputs such as annotations without adjusting treatment parameters or accepting the treatment plan, and the annotations are mirrored on both the first display 140 and the second display 125, such that the annotations can be viewed on both displays without allowing control of the surgical procedure or surgical input parameters from the second display 125. The annotations provided with input to the second display 125 can be helpful to communicate with the surgeon and can be used for telestration and tele-proctoring, for example. Although reference is made to a first display and a second display, additional displays can be provided. For example a remote display can be configured to allow annotations that are viewed on displays 125 and 140, which can allow remote telestration and remote proctoring, for example. In some embodiments, the second display 125 comprises a remote display, such as a display in another room, another building, another state, or another country, for example.

In some embodiments, the control panel 302 provides one or more instructions 304 to the user. The instructions may comprise any suitable instruction, such as an instructions to be perform at next step at a current stage in the procedure, or an instruction to view a video, for example.

In some embodiments, the control panel comprises a navigation bar 330. The navigation bar may comprise icons for the user to navigate through stages of the procedure. In some embodiments, the navigation bar 330 comprises a previous icon for the user to go back to a prior screen and step of the procedure, and a next icon for the user to advance to a next screen and step of the procedure.

In some embodiments, the navigation bar 330 comprises one or more indicators for a user to assess progress of the navigation through different stages of the procedure. In some embodiments, the user navigation bar 330 is configured to display a plurality of modes 340 of the system 100, such as one or more of a set up mode, a plan mode or a treatment mode. Each of the modes may comprise a plurality of sub-modes, in which each of the corresponding sub-modes corresponds to a stage of the associated mode. In some embodiments, the setup mode comprises one or more of a TRUS sub-mode to insert the ultrasound probe such as a TRUS probe, a handpiece mode for the user to insert the treatment mode with the handpiece, and an align mode for the user to align the treatment probe and the TRUS probe with each other to visualize the treatment probe with the TRUS probe. In some embodiments, the plan mode comprises an angle and depth sub-mode for the user to determine a plurality of angles and depths of treatment from the probe on a plurality of transverse images, a registration sub-mode for the user to register the position of the energy source along a longitudinal image such as a sagittal image, and a profile sub-mode for the user to adjust markers associated with a positions of the treatment plan on the longitudinal image such as a sagittal image.

Generally, an icon of the user interface 103 is highlighted it appears differently relative to other icons, for example if its contrast, boldness, color or shape on the display relative to other icons is different than the contrast, boldness, color or shape of other, non-highlighted icons. In some embodiments, a highlighted icon comprises an icon that is more visible than non-highlighted icons.

Referring to FIG. 3A, the set up mode is highlighted relative to other modes of the plurality of modes 340, such as relative to the plan mode icon and the treat mode icon. The handpiece sub-mode 343 is highlighted relative to the other sub-modes of the plurality of sub-modes 342, such as the TRUS sub-mode and the Align sub-mode.

Although reference is made to the plurality of modes and corresponding sub-modes in the context of a navigation bar, these modes and corresponding sub-modes can be presented on the display outside the navigation bar.

In some embodiments, the UI 103 is configured for the user to press the next icon 334 to proceed to the next stage of the procedure, although the user may press the previous icon 332 to proceed to a prior stage of the procedure.

Referring to FIGS. 3B and 3C, the UI 103 is configured to align the treatment probe 150 and ultrasound device such as TRUS probe 160.

As shown in FIG. 3B, the primary image 312 comprises a longitudinal ultrasound image such as sagittal image from TRUS probe 150, and the secondary image 314 comprises the endoscope image such as cystoscope image. The one or more modes 340 comprises the setup mode and the one or more sub-modes 342 comprises the align mode with the align sub-mode highlighted in relation to the other sub-modes 342, such as the hand piece mode 343. One or more markers 303 shows a position of the display corresponding to an end of the ultrasound probe such as TRUS probe 160. In some embodiments, the path of the energy 306 from the energy source is visible on the primary image 312 shown on the display.

In some embodiments, the processor is configured with instructions to automatically change the source of the primary image 312 shown on the display in accordance with the current task to be performed by the user, for example a task associated with the current mode of the one or more modes 340 and the current sub-mode of one or more sub-modes 342. As shown in FIG. 3B, the UI has changed the primary image 312 to a real time ultrasound image and the secondary image 314 to the real time endoscope image. In some embodiments, the user interface is configured to change the primary image 312 from the real time endoscopic image such as the cystoscope image to the real time ultrasound image, for example to a longitudinal image such as the sagittal image in response to the stage of treatment progressing from the handpiece sub-mode 343 to the alignment sub-mode. In some embodiments, the processor is configured to automatically change the primary image 312 of the user interface 103 in response to the user progressing to another stage of a mode or sub-mode, such as changing the primary image from a longitudinal image such as a sagittal image, for example. In some embodiments, the secondary image 314 comprises the real time endoscope image such as the cystoscope image while the user interface or the user changes the ultrasound image shown as the primary image 312.

In some embodiments, the one or more user instructions 304 are configured to instruct the user. The one or more user instructions 304 may comprise a single displayed instruction or a plurality of instructions, for example. In some embodiments, the user is instructed to press on the foot pedal to release energy from the energy source, in order to detect the position of the energy source such as a water jet, although other energy sources can be detected on the primary image as described herein. In some embodiments, the user is instructed to translate the imaging device such as the TRUS probe along a longitudinal axis of the imaging probe to align the one or more markers 303 with the energy source such as the water jet. In some embodiments, the user is instructed to provide an input such as a click or touch on the information icon 328 to obtain additional information about the current mode and sub-mode, for example. In some embodiments, the user is instructed to change the view from the imaging device from a longitudinal mode such as a sagittal mode to a transverse mode.

In some embodiments, the UI 103 is configured for the user to press the next icon 334 to proceed to the next stage of the procedure, although the user may press the previous icon 332 to proceed to a prior stage of the procedure.

As shown in FIG. 3C, the primary image 312 comprises a transverse ultrasound image such as transverse image from TRUS probe 150, and the secondary image 314 comprises the endoscope image such as cystoscope image. The one or more modes 340 comprises the setup mode and the one or more sub-modes 342 comprises the align mode with the align sub-mode highlighted in relation to the other sub-modes 342, such as the hand piece mode 343. One or more markers 303 shows a position of the display corresponding to an end of the ultrasound probe such as TRUS probe 160. In some embodiments, path of energy 306 from the energy source is visible on the primary image 312 shown on the display.

In some embodiments, the processor is configured with instructions to automatically change the primary image 312 shown on the display in accordance with the current task to be performed by the user. As shown in FIG. 3C, the UI has automatically changed the primary image 312 to a real time ultrasound transverse image from the real time longitudinal image as shown in FIG. 3B, while the secondary image 314 remains the real time endoscope image.

The one or more user instructions 304 are configured to instruct the user at this stage of the align sub-mode. In some embodiments, the user is instructed to press on the foot pedal to release energy from the energy source, in order to detect the position of the energy source such as a water jet, although other energy sources can be detected on the primary image as described herein. In some embodiments, the user is instructed to rotate the imaging device such as the TRUS probe about a longitudinal axis of the probe to position a hyperechoic shadow of the treatment probe at a desired position, such as 12 o'clock, which corresponds to a substantially vertical position above the imaging probe as shown in the image, e.g. to within about 10 degrees of vertical. In some embodiments, the user is instructed to provide an input to the foot pedal to visualize the position and orientation of the energy 306 from energy source.

In some embodiments, the user is instructed to rotate the handpiece about the longitudinal axis of the probe and the handpiece, for example to rock the handpiece, such that the orientation of the energy source such as a water jet comprises a substantially vertical orientation, e.g. to within about 10 degrees of vertical, although any suitable orientation can be used. In some embodiments, the positioning of the display above the probes facilitates alignment of the probes, for example with the active light emitting area of the display oriented such that the display is substantially parallel to the transverse image and the longitudinal axis of one or more of the probes substantially perpendicular to the active light emitting area of the display as described herein. In some embodiments, the user is instructed to provide an input such as a click or touch on the information icon 328 to obtain additional information about the current mode and sub-mode, for example. In some embodiments, the user is instructed to change the view from the imaging device from a longitudinal mode such as a sagittal mode to a transverse mode.

In some embodiments, the UI 103 is configured for the user to press the next icon 334 to proceed to the next stage of the procedure, although the user may press the previous icon 332 to proceed to a prior stage of the procedure.

Referring to FIG. 3D, the user interface 103 can be configured to receive user input to plan the treatment with a plurality of images that are transverse to the probe 150, such as a plurality of transverse images from the TRUS probe. In some embodiments, the mode of the one or more modes 340 comprises a plan mode. In some embodiments, the plan mode comprises a plurality of sub-modes 342, in which the plurality of sub-modes comprises an angle and depth mode, a registration mode, and a profile mode. As shown in FIG. 3D, the plurality of modes 340 comprises the plan mode and the plurality of sub-modes 342 comprises an angle and depth mode 346. In some embodiments, the UI is configured to highlight the current mode such as the plan and the sub-mode such as the angle and depth mode 346. With the angle and depth sub-mode 346, the user is able to adjust the angle of treatment and the radial distance of the treatment from the probe such as a depth of treatment from the probe 150.

With the plan mode and the angle and depth sub-mode, the user is provided with an instruction 304 to select an anatomical tab and adjust the plurality of markers 353.

In some embodiments, in the plan mode the user interface 103 is configured for the user to provide input related to the angles of treatment delivered with the energy source such as a directional energy source on the treatment probe 150. In some embodiments, the primary image 312 comprises a transverse image such as a transverse ultrasound image, and the secondary image 314 comprises an endoscope image such as a cystoscope image. The image display area 310 shows the primary image and the secondary image. A plurality of markers 353 is, shown overlaid on the primary image. The plurality of markers 353 is configured to allow the user to adjust the treatment.

In some embodiments, the primary image 312 shown on the display comprises an image among a plurality of images at different tissue depths, and the user interface 103 is configured for the user to select the primary image to display among a plurality of images along image planes from different depths of the tissue, such as transverse images from different longitudinal locations along probe 150. A plurality of planning tabs 390 is shown above the primary image shown on the display, which allows the user to select the image to view for planning the treatment, although the plurality of planning tabs may be displayed at other locations. The plurality of planning tabs may comprise a first tab corresponding to a first depth along a longitudinal axis of the probe, such as a median lobe of the prostate, a second tab corresponding to a second longitudinal depth along the probe, such as a bladder neck, and a third tab corresponding to a second longitudinal depth along the probe, such as a mid-prostate, for example. The plurality of markers 353 is shown on each of the plurality of images selected with a corresponding tab.

In some embodiments, the plurality of markers 353 are shown at initial positions for each of the plurality of planning tabs and subsequently adjusted by the user. The initial positions of the plurality of markers may comprise default values, or values suggested with an artificial intelligence (AI) algorithm. In some embodiments, the UI is configured to display an assisted planning icon 399 for the user to turn on assisted planning. In some embodiments, the assisted planning is configured to indicate one or more tissue boundaries with a marker, such a dashed line. In some embodiments, the tissue boundary comprises a prostate boundary such as a boundary of the prostate. Alternatively or in combination, the assisted planning may provide initial locations for the plurality of markers 353 on the display, in which the locations of the plurality of markers 353 correspond to locations determined with the AI algorithm.

In some embodiments, a reset input 396 is shown with an icon and configured for the user to reset the markers shown in the currently selected tab to previous locations that the user has entered, a default locations, or locations determined with the AI algorithm.

The UI is configured for the user to plan the treatment at a plurality of depths with the markers 353 shown on each of the plurality of images corresponding to different depths in the tissue. This allows the user to plan the treatment angles at different depths of the tissue. In some embodiments, the user selects the planning tab corresponding to an image, and adjusts the locations of the one or more markers 353 on the image shown with the corresponding planning tab.

The UI 103 is configured for the user to select a marker among the plurality of markers 353 to adjust the location of the selected marker on the primary image 312. In some embodiments, the UI comprises a plurality of marker select icons such as buttons 350, which allows the user to select a marker to adjust among the plurality of markers 353. In some embodiments, the plurality of markers 353 comprises a first marker (1), a second marker (2), and a fourth marker (4). With a marker selected, the user is able to adjust the position of the selected marker on the primary image 312, which results in a corresponding change to one or more of the depth of treatment from the probe or the angular location of the treatment. The selected marker among the plurality of markers can be moved with user input in any way. In some embodiments, the display comprises a touch screen display, in which the UI 103 is configured for the user to drag the marker to a desired location. Alternatively or in combination, the marker can be moved with an input to directional icon, such as directional pad 374, for example. In some embodiments, the UI is configured for the user to make a coarse adjustment to the selected marker shown on the display by dragging the marker to a different location on the touch screen display, and to make a fine adjustment by pressing a button on the directional icon such as directional pad 374. In some embodiments, the directional pad is configured to move the location of the selected marker by an incremental amount in response to the user depressing a directional icon, such as an amount within a range from about 0.05 mm to about 0.2 mm in response to the user pressing on the directional icon, for example. In some embodiments, the amount of movement corresponds to a scale of the image shown on the display, such as a scale in millimeters (mm).

As shown in FIG. 3D, for example, marker three (3) has been selected and the user is allowed to adjust the position of marker three. The selected marker can be highlighted on the display, for example with a color, or other indication such as geometric shape around the marker. The geometric shape may comprise any suitable shape such a bracket, circle, square or diamond, for example.

In some embodiments, the UI 103 is configured is configured to constrain the direction of movement of the selected marker and to indicated to the user that the direction of movement of the marker has been constrained, for example by highlighting the allowable directions on the icon. As shown in FIG. 3D, the movement of the selected marker among the plurality of markers is limited to up and down movement on the display, and the directional icon such as direction pad 374 shows the up and down arrows highlighted with respect to the lateral arrows.

In some embodiments, the UI 103 is configured for the user to select each of the plurality of markers, e.g. markers 1, 2, 3 and 4, and to adjust the position of each of the selected markers. For example, the user can select marker 1 to align the treatment profile with the image of the treatment probe 150 shown in the primary image 312. In some embodiments, the directional icon such as directional pad 374 shows four directional icons highlighted, e.g. left, right, up and down, to indicate to the user that the marker can be moved in each of the corresponding directions. The user can select marker 2 to adjust the depth of the treatment. The user can select marker 3 to adjust an angle of treatment and a depth of the treatment with respect to treatment probe 150 corresponding to the position of marker 1. In some embodiments, the directional icon such as directional pad 374 shows four directional icons highlighted, e.g. left, right, up and down, to indicate to the user marker 3 can be moved in each of the corresponding directions. The user can select marker 4 to adjust an angle of treatment and a depth of the treatment with respect to treatment probe 150 corresponding to the position of marker 1. In some embodiments, the directional icon such as directional pad 374 shows four directional icons highlighted, e.g. left, right, up and down, to indicate to the user marker 4 can be moved in each of the corresponding directions. The adjustment to markers 3 and 4 can be used to adjust the planned angle of treatment 392, which corresponds to an angle between marker 3 and marker 4. The planned angle of treatment 392 can be shown on the display, and may comprise any suitable angle, such that the user can adjust the displayed angle of treatment 392 from 145.5 degrees to another angle in response to user preferences.

If the user is not satisfied with a position of the selected marker after adjustment, the user can select the undo icon 351 with a user input such as touching the display at the location of the undo icon, which will move the marker to a location of the marker prior to user adjustment.

In some embodiments, the UI 103 is configured for the user to press the next icon 334 to proceed to the next stage of the procedure, although the user may press the previous icon 332 to proceed to a prior stage of the procedure.

Referring to FIG. 3E, the user interface can be configured to receive user input to plan the treatment with one or more longitudinal images such as sagittal images. The one or more longitudinal images is aligned with treatment probe 150, such that treatment probe 150 extends a distance along the one or more longitudinal images. The one or more longitudinal images may be acquired with the TRUS probe, an ultrasound probe located on the treatment probe 150, or an external probe coupled to a skin of the patient, for example. As shown in FIG. 3E, the plurality of modes 340 comprises the plan mode and the plurality of sub-modes 342 comprises the profile mode 344. In some embodiments, the UI is configured to highlight the current mode such as the plan mode and the sub-mode such as the profile sub-mode 344. With the profile sub-mode 344 highlighted, the user is able to adjust the distance from the treatment probe, such as a depth of treatment from the probe 150.

In some embodiments, in the plan mode the user interface 103 is configured for the user to provide input related to the depth and longitudinal position of treatment delivered with the energy source such as a directional energy source on the treatment probe 150. In some embodiments, the primary image 312 comprises the longitudinal image such as a sagittal ultrasound image, and the secondary image 314 comprises an endoscope image such as a cystoscope image. A plurality of markers 353 is, shown overlaid on the primary image. The plurality of markers 353 is configured to allow the user to adjust the treatment.

In some embodiments, the UI 103 is configured for the user to perform a registration step at a registration stage of the work flow. The registration step may be performed as part of the planning mode of the one or more modes 340. In some embodiments, the user performs the registration step at a registration sub-mode of the one or more sub-modes 342. In some embodiments, the registration sub-mode is performed between the angle and planning sub-mode and the profile sub-mode, although the registration can be performed at any suitable stage of the procedure, for example with the alignment stage.

In some embodiments, the user views the treatment probe with a longitudinal image such as a sagittal image, such that the treatment probe 150 extends along a plane of the image. With the registration sub-mode, the user indicates a location of the energy source with a user input with the energy source located at a distal position. In some embodiments, the user inputs a second location corresponding to a proximal location of the energy source. In some embodiments, the user inputs the proximal location on the display. While the proximal location of the energy source can be input in many ways, in some embodiments the user places a marker along a pathway of the energy source, for example at a location along the probe. Alternatively or in combination, the user can move the energy source to the proximal location and input the proximal location. In some embodiments, the energy source such as a water jet is activated at the first location to allow the user to accurately identify the position of the energy source shown on the display and provide an appropriate input at the location. In some embodiments, the first location and the second location are shown with a line extending therebetween, and the user is allowed to adjust one or more of the first location or the second location with a coarse or fine input to the display, for example with the directional pad 374, for example. The first location and the second location can be used to register the plurality of transverse images with the position of the probe, which can be helpful for generating a treatment plan such as a 3D treatment plan.

As shown in FIG. 3E, the treatment probe 150 is shown on the image with a plurality of markers 353. In some embodiments, a longitudinal scale 380 is shown with a radial scale from the longitudinal axis of the probe such as a depth scale 382. The primary image 312 is with a plurality of markers 353 for the user to adjust the treatment on the longitudinal image. In some embodiments, one or more treatment limits 355 is show on the display to show the maximum depth of treatment. In some embodiments, the plurality of markers shown on the display correspond to plurality of regions of tissue to be protected. The markers may correspond to any tissue, such a verumontanum (veru), which can be identified veru protection zone (VPZ) marker 362, or a bladder neck (BN) identified with a BN marker 364. Each of the plurality of protection zones can be adjust with a user input to a corresponding icon. In some embodiments, the treatment at the VPZ can be adjusted with an input to a VPZ icon 366. In some embodiments, the treatment at the BN can be adjusted with an input to a BN icon 368.

With the plan mode and the profile sub-mode, the user is provided with an instruction 304 to adjust the plurality of markers 353 as needed for treatment.

In some embodiments, the UI 103 is configured with the plurality of selection markers 350, in which a user can select a marker to be adjusted as described herein. The selected marker can be adjusted with coarse or fine adjustment as described herein. In some embodiments, the starting location of the treatment is selected for adjustment with a treatment start (TS) icon 352. Once the TS icon 352 has been selected, the location of the start of the treatment can be adjusted with coarse or fine adjustment as described herein. In some embodiments, the ending location of the treatment is selected for adjustment with a treatment end (TE) icon 354. Once the TE icon 354 has been selected, the location of the end of the treatment can be adjusted with coarse or fine adjustment as described herein. In some embodiments, the user interface 103 comprises a plurality of user selectable markers 353, such as a first selectable marker (1), a second selectable marker (2), a third selectable marker (3), a fourth selectable marker (4) and a fifth selectable marker (5). Each of the plurality of markers can be selected with a user input to a corresponding icon among the plurality of icons 350, and the location of the corresponding marker adjusted on the primary image 312, for example with coarse or fine movement as described herein. For example a first marker can be selected with a first input to a first icon 356, such as a user touching the first icon 356. Additional markers can be selected and adjusted as described herein.

As shown in FIG. 3E, for example, marker four (4) has been selected and the user is allowed to adjust the position of marker four, for example with coarse or fine adjustment as described herein. The selected marker can be highlighted on the display, for example with contrast, a color, or other indication such as geometric shape around the marker as described herein. The geometric shape may comprise any suitable shape such a bracket, circle, square or diamond, for example. The directional icon such as directional pad 374 can be highlighted to indicate the allowable movements of the marker, for example.

The plurality of treatment markers shown in the plurality of transverse images and the one or more longitudinal images such as one or more sagittal images can be used to generate a three-dimensional treatment plan.

In some embodiments, the UI 103 is configured for the user to press the next icon 334 to proceed to the next stage of the procedure, although the user may press the previous icon 332 to proceed to a prior stage of the procedure.

Referring to FIG. 3F, the UI 103 is shown in a treatment mode. A treat mode 347 is shown highlighted among the plurality of modes 340, and the treatment sub-mode is show highlighted. The primary image is shown on the display with a locked icon 397 to indicate that the treatment plan has been accepted and locked. The primary image and the secondary image are shown on the display as described herein. The user instruction 304 shows an instruction for the user to begin treatment by pressing and holding the foot pedal and to pause treatment by releasing the foot pedal. In some embodiments, the instruction comprises an instruction not to move the TRUS probe or the handpiece, for example. In some embodiments, the longitudinal image such as a sagittal image is shown as the primary image on the display. With the treatment mode, information related to the treatment is shown on the display, such as an intended position of the energy source along the longitudinal axis of the probe overlaid on the image. In some embodiments, the longitudinal scale 380 and the radial scale such as depth scale 382 are shown on the longitudinal image such as the sagittal image during treatment.

Although FIGS. 3A to 3F show a user interface in accordance with some embodiments, one of ordinary skill in the art will recognize many variations. For example, some of the screens may be modified, the order of the screens can be provided in sequence as shown or the order changed. Some of the screens may not be used and some of the screens can be modified in accordance with the present disclosure. Some of the modes may comprises sub-modes of other modes, and some of the sub-modes may comprises one or more modes with additional sub-modes.

The system 100 can be configured in many ways in accordance with the present disclosure. In some embodiments, the one or more processors of the system 100 is configured to provide one or more safety features. In some embodiments, the one or more processors is configured to prime the energy source initially at a first flow rate with a first source of saline prior to insertion of the treatment probe into the patient, and the processor is configured to decrease the flow rate to the energy source to prime the energy source a second time at a second flow rate less than the first flow rate with a second source of saline if the first source has been depleted with the treatment probe inserted into the patient. In some embodiments, the system is configured to prime the energy source such as a water jet emitted from the probe 150. In some embodiments the system is configured to prime the energy source at over 90% of the treatment flow rate initially, e.g. at 100% of the treatment flow rate and to provide instructions to the user to prime the water jet with the water jet outside the patient. If system runs low on saline or out of saline to the energy source after the initial priming and prior to completion of treatment, the one or more processors of the system 100 are configured to automatically reduces the priming of the energy source to no more than 80%, for example to 50% or less. While this safety feature can be provided in many ways, in some embodiments the system is configured to automatically reduce the priming flow rate if the system runs low on saline or out of saline after the probe such as the TRUS probe has been inserted into the patient.

In some embodiments, the treatment probe comprises a handpiece and a motor-pack, and the one or more processors is configured to detect decoupling of the motor-pack from the handpiece. The processor is configured to disable treatment and direct the user to repeat one or more of an alignment mode or a planning mode of treatment prior to allowing the user to resume treatment, for example. In some embodiments, if the motor-pack and handpiece have become disconnected, the system is configured to bring the user back to the handpiece step, e.g. the handpiece sub-mode of the plurality of modes, for the user to complete one or more previously completed steps such as alignment and the planning steps. In some embodiments, the user interface is configured force the user to complete one or more of these previously completed steps.

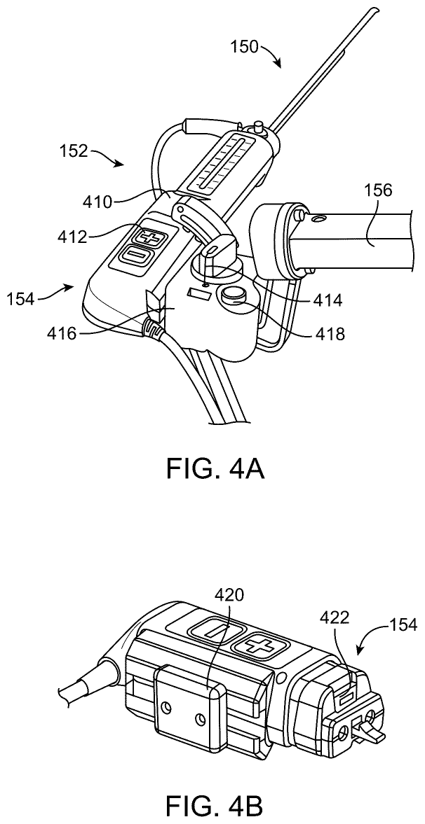

FIG. 4A shows a treatment probe 150 configured for use with a surgical system. The treatment probe 150 comprises a distal portion sized for insertion into the patient. In some embodiments, the treatment probe comprises a handpiece 152 configured to manipulate the probe during insertion of the distal end into the patient. In some embodiments, the handpiece 152 is configured to couple to one or more actuators such as motors configured to move the treatment probe in response to computer control as described herein. In some embodiments, the handpiece is configured to couple to a motor-pack 154, which is configured to move an energy source such as directional energy source to a plurality of positions and orientations as described herein. In some embodiments, the handpiece 152 comprises a handpiece release button 410 configured to release the handpiece 152 from the motor-pack 154. In some embodiments, the motor-pack comprises one or more controls 412 to control an amount of energy of the energy source, such as an amount of waterjet power to a water jet.

While the handpiece 152 can be configured in many ways, in some embodiments, the handpiece 152 comprises a linkage to rotate and translate the energy source in response to movements such as rotational movements transmitted from the motor-pack.

While the arm 156 that supports treatment probe 150 can be configured in many ways, in some embodiment the arm comprises lever and a motor-pack release knob 414 configured to release the motor-pack from arm 156, and to securely couple the motor-pack to the arm. While motor-pack 154 can be coupled to the arm 156 in many ways, in some embodiments, the arm 156 comprises a magnetic mount 416 configured to couple to the motor-pack to the arm. In some embodiments, the arm comprises a leveling button configured for a user to level the motor-pack, for example by pressing the button and then releasing the button to lock in the angle of inclination of the motor-pack, e.g. to level the motor-pack and the probe.

FIG. 4B shows the motor-pack 154 configured to couple to the handpiece 152 of treatment probe 150 and the arm 156. In some embodiments, the motor-pack 154 comprises a mounting plate 420 configured to couple to the arm. In some embodiments, the motor-pack 154 comprises a handpiece interface 422 configured to interface the motor-pack with the handpiece and may comprise one or more gears and an electrical connector to couple to the handpiece.

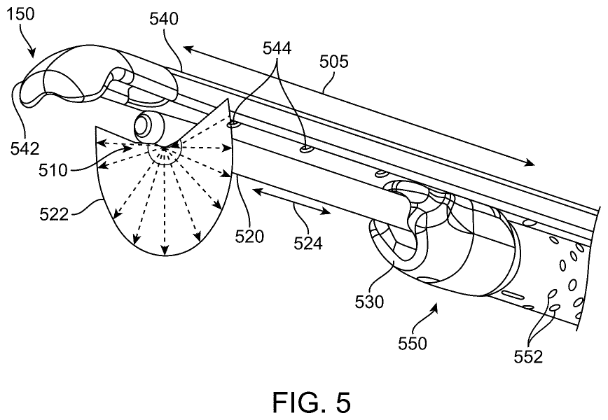

FIG. 5 shows treatment probe 150 and corresponding movements of an energy source 510. In some embodiments, the energy source 510 is carried on a carrier 520 that is configured to carry the energy source move the energy source. In some embodiments, the carrier 520 is configured to carry the energy source to a plurality of angles and longitudinal positions corresponding to a treatment plan such as a three dimensional treatment plan as described herein. In some embodiments, energy source 510 is configured to rotate to a plurality of angles 522 in accordance an angle of the treatment plan as described herein. In some embodiments, the energy source is configured to translate as shown with arrows 524. The rotational movement and translational movement of the energy source can be combined in accordance with the 3D treatment plan as described herein, for example.

The energy source 510 may comprise any suitable energy source, such as such as one or more of an electrode, a loop electrode, a laser source, a thermal energy source, a mechanical energy source, a mechanical sheer, an ultrasound probe, a cavitating ultrasound probe, a water jet, e.g. a fixed pressure water jet, a plasma source, a steam source, a morcellator, a trans urethral needle, a photo ablation source, a radiation energy source, a microwave energy source or a water jet evacuation source, for example.

While the treatment probe 150 can be configured in many ways, in some embodiments the treatment probe comprises an opening 530 configured to receive an endoscope such as a cystoscope to view the treatment area. In some embodiments, the probe 150 comprises a support portion 530 that is configured to support the carrier and the endoscope. The support portion 530 may comprise one or more openings 552 configured to release a fluid such as saline from a saline source as described herein. In some embodiments, the probe comprises a portion 540 comprising sufficient stiffness to advance the probe into the patient. In some embodiments, the stiff portion 540 extends to a curved distal end 542 to allow the probe to be inserted into tissue, such as along a lumen. In some embodiments, the stiff portion 540 comprises one or more openings 544 to remove material such as fluids from the surgical site. In some embodiments, the one or more openings 544 is coupled to an evacuation pump such as the aspiration pump as described herein.

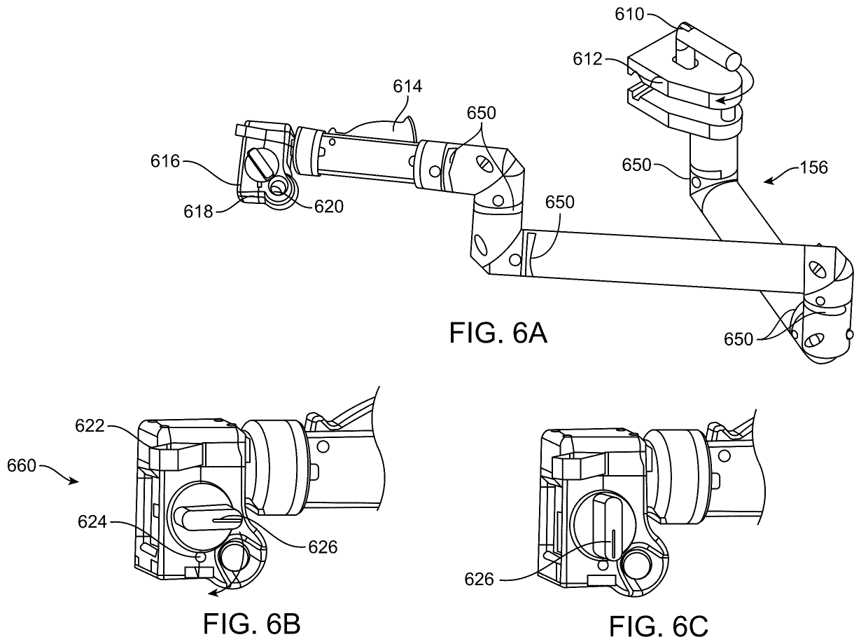

FIG. 6A shows an arm 156 configured to support the treatment probe 150. The arm comprises a bedrail clamp 612 configured to couple to a bedrail of the patient support as described herein. The arm 156 comprises a clamp lever 610 configured to securely couple the bedrail clamp 610 onto the bedrail of the patient support. In some embodiments, the arm comprises an articulation release lever 614 configured to allow the arm to articulate to adjust the position of the arm. The arm may comprise a plurality of locking joints 650 that are configured to move freely when the articulation release lever 614 is pressed down by a user, and then locked in place when the lever is released. The joints of the arm may comprise locking joints, such as joints with clutches, or magnetic joints, that can be moved into position prior to surgery and lock into place during surgery with computer controlled movement of the probe as described herein. Although reference is made to a manually manipulated arm, in some embodiments the arm comprises a robotic arm.

FIG. 6B shows mounting device 660 configured to couple to the motor-pack, in which the mounting device 660 comprises an unlocked configuration. The mounting device 660 comprises a rotation guide rail 622, a mounting lock indicator 624, and a motor-pack release knob 626. As shown in FIG. 6B, the release knob 626 is shown in an unlocked configuration.

FIG. 6C shows the mounting device of FIG. 6B, in which the release knob 626 comprises a locked configuration, in in which the motor-pack release knob has been rotated to a locked configuration.

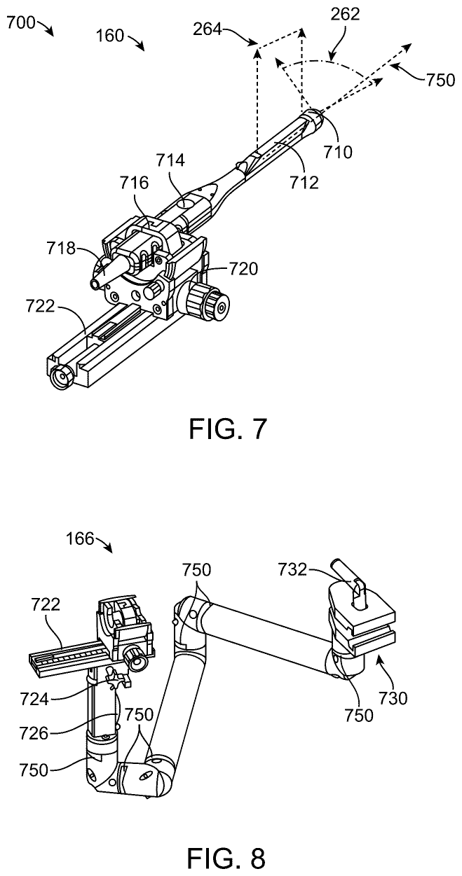

FIG. 7 shows an ultrasound imaging device 700 to image tissue of the patient. The imaging device comprises one or more arrays to image tissue of the patient. The imaging device 700 may comprise an ultrasound imaging array, and may comprise a one dimensional (1D), a two dimensional (2D) or a three dimensional (3D) imaging device. In some embodiments the one or more arrays comprises a transverse array 710 configured to image tissue along a plane that is transverse to an elongate axis of the probe 750. The probe 160 extends in an elongate direction along an axis 750 to a distal tip sized for inversion into a patient. The transverse array 710 extends in a direction transverse to the longitudinal axis 750 of the probe 160 to generate images that are transverse to the longitudinal axis 750, for example perpendicular to the axis 750. In some embodiments, the ultrasound imaging device is configured to generate transverse images within a field of view 262. In some embodiments, the ultrasound imaging device comprises a longitudinal array of transducers, such as a sagittal array 712 of transducers. The longitudinal array of transducers 712 extends in a longitudinal direction along probe 150 corresponding to axis 750. In some embodiments, the longitudinal array of transducers 712 is configured to generate a longitudinal image of tissue within a longitudinal image field of view, such as a sagittal plane image field of view 264.

In some embodiments, the ultrasound imaging device comprises a T-shaped marking to allow the user to identify the longitudinal axis of the array and the transverse axis of the array. In some embodiments, the transverse array 710 corresponds to a transverse imaging plane of the T-shaped marking, e.g. the top of the T-shaped marking, and the and the longitudinal array such as sagittal array 712 corresponds to the longitudinal imaging plane of the T-shaped marking, e.g. the bottom part of the T-shaped marking.

In some embodiments, the imaging device 700 comprises a probe configured to be inserted into the patient, such as a TRUS probe 160. In some embodiments, the TRUS probe 160 comprises plane switch button 714 to toggle between longitudinal and transverse images shown on the display as described herein. In some embodiments, the TRUS probe comprises a probe cradle and latch 716 configured to couple the probe 150 to a stepper 722. The stepper comprises a knob 720 that allows the stepper to advance and retract the probe from the patient. The stepper is configured to couple to the TRUS arm as described herein and move the probe 160 while the arm remains fixed, for example with rotation of the knob 720. By rotating the knob to a plurality of positions, the probe is placed at a plurality of locations corresponding to a plurality of depths of the transverse images, which can be used for treatment planning as described herein. In some embodiments, the TRUS probe 160 comprises a cable 718 extending from a proximal portion of the TRUS probe 160.

In some embodiments, the location of the one or more of the longitudinal array such as sagittal array 712 or the transverse array 710 of the ultrasound device 700 is visible to the user. In some embodiments, the one or more of the longitudinal array or the transverse array is covered with a material which is visibly different from other areas of the distal end of the probe, such as with a different color or shading for example. The visible portion of the probe corresponding to the locations of the one or more transducer arrays can be associated with the position of the display. In some embodiments, the transverse array is approximately parallel to an active light emitting area of the display, and the visible portion of the transverse array can help the user associate the physical location of the tissue shown in transverse images with the location and orientation of the transverse array 710. In some embodiments, the longitudinal array such as the sagittal array 712 is approximately perpendicular to an active light emitting area of the display, and the visible portion of the longitudinal array can help the user associate the physical location of the tissue shown in longitudinal images such as sagittal images with the location and orientation of the longitudinal array such as sagittal array 712. In some embodiments, the ultrasound imaging device comprises TRUS probe 160, in which the locations of transverse array 710 and the longitudinal array such as sagittal array 712 are visible to the user in relation to the longitudinal axis 750 of the probe, for example.

FIG. 8 shows an arm 166 configured to support an ultrasound imaging device as in FIG. 7. The arm comprises a bedrail clamp 730 is configured to couple to a bedrail of the patient support as described herein. The arm 166 may comprises a clamp lever 732 configured to securely couple the bedrail clamp 730 onto the bedrail of the patient support. In some embodiments, the arm comprises an articulation release lever 726 configured to allow the arm to articulate to adjust the position of the arm. The arm may comprise a plurality of locking joints 750 that are configured to move freely when the articulation release lever 726 is pressed down by a user, and then lock in place when the lever is released. The joints of the arm may comprise locking joints, such as joints with clutches, or magnetic joints, that can be moved into position prior to surgery and locked into place during surgery with computer controlled movement of the probe as described herein. Although reference is made to a manually manipulated arm, in some embodiments the arm comprises a robotic arm.

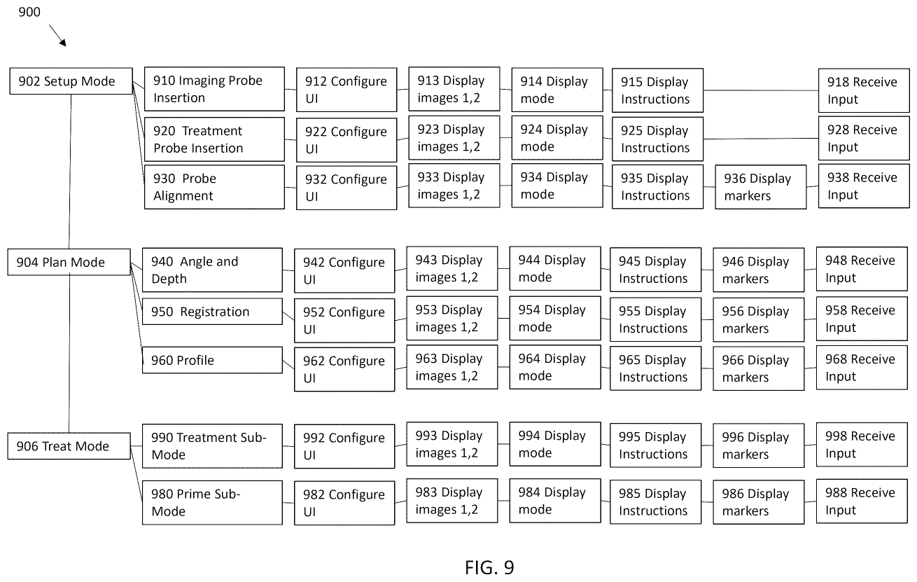

FIG. 9 shows a user interface method 900. The method 900 may utilize one or more components of the system and features of the user interface as described herein.

At a setup mode 902, the user interface is configured to set up the system for surgery, for example with an insertion mode, in which the system is configured to insert a probe into a patient, for example as described herein.

At a plan mode 904, the user interface is configured with a plan mode to allow a user to plan a treatment, for example as described herein.

At a treatment mode 906, the user interface is configured with a treat mode, for example as described herein.

Each of modes 902, 904 and 906 may comprise or be associated with additional steps and modes or sub-modes, for example as described herein. Further, each of these steps, modes, or sub-modes may correspond to one or more configurations and processes of the system as described herein. Any of these modes can be configured to provide one or more features of the user interface as described herein, for example with reference to FIGS. 3A to 3F.

In some embodiments, the instructions implemented with the processor are configured to automatically select a primary image source for a primary image display area and a secondary image source corresponding to a secondary image display area in response to the current mode and the current sub-mode of the system, which can make it easier for the user to proceed among the different modes and sub-modes of the user interface.

In some embodiments, the setup mode 902 comprises one or more modes or sub-modes, such one or more of as an imaging probe insertion mode 910, a treatment probe insertion mode 920 or a probe alignment mode 930. In some embodiments, one or more of imaging probe insertion mode 910, treatment probe insertion mode 920 or probe alignment mode 930 comprises a sub-mode of another mode, such as setup mode 902.

In some embodiments, the plan mode 904 comprises one or more modes or sub-modes, such as one or more of an angle and depth mode 940, a registration mode 950, or a profile mode 960.

In some embodiments, the treatment mode 906 comprises one or more modes or sub-modes such as one or more of a treatment mode 990 or a prime mode 980.

In some embodiments, the imaging probe insertion mode 910 comprises one or more of a user interface configuration step 912, a display primary and secondary images step 913, a display current mode step 914, a display instructions step 915, or a receive user input step 918. In some embodiments, the imaging probe insertion mode 910 configures the UI for an imaging probe to be inserted into the patient. In some embodiments, the imaging probe comprises an ultrasound probe such as a TRUS imaging probe as described herein.

At the user interface configuration step 912, one or more features of the user interface is displayed, such one or more of the user input features, the image display area, the primary image, the secondary image, the tool bar, the control panel, the displayed mode, the displayed sub-mode, the navigation bar as described herein, for example with reference to FIG. 3A. In some embodiments, the instructions executed by the one or more processors are configured to provide the one or more features of the configuration step 912 in response to a user input such as a selection indicating completion of a prior mode or sub-mode, e.g. the next icon 334 as described with reference to FIGS. 3A to 3F.

At the display primary and secondary images step 913 of the imaging probe insertion mode 910, the user interface is configured to automatically display a real-time image from the ultrasound probe in the primary image display area. In some embodiments, the primary image display area comprises a real time transverse ultrasound image or a real time longitudinal ultrasound image, such as a sagittal image, and the user interface is configured for the user to toggle between the real time transverse image and the real time longitudinal image being shown in the primary image display area with a user input as described herein.

In some embodiments, the user interface is configured to automatically display an image from the ultrasound probe in the primary image display area and an image from the endoscope or other imaging device in the secondary image display area during the display primary and secondary images step 913 of the imaging probe insertion mode 910. In some embodiments, the user interface is configured to toggle between a transverse ultrasound image and a longitudinal, e.g. sagittal, ultrasound image being shown in the primary image display area, and the secondary image display area, depending on the ultrasound imaging probe inserted into the patient during imaging probe insertion mode 910, for example.

At a display current mode step 914 of the imaging probe insertion mode 910, the user interface is configured to display the current mode of the system such as the set up mode 902 and optionally a current sub-mode, such as the insert imaging probe mode 910. In some embodiments, the displayed set up mode 902 is highlighted with relative to other displayed modes as described herein, e.g. modes 904 and 906. In some embodiments, the current insert imaging probe insertion sub-mode 910 is displayed with the current mode 902 as described herein. In some embodiments, the current sub-mode 910 is highlighted with respect to other sub-modes 920, 930, and these are displayed with the current mode 910. At a display user instructions step 915, the user interface is configured to automatically display one or more instructions for the user to insert the ultrasound treatment probe and optionally display the one or more instructions in a user input area of the control panel, for example. In some embodiments, the one or more instructions to the user comprise instruction to 1) in transverse view, insert the ultrasound probe into the patient, ensure the prostate is centered, and with appropriate amount of compression, advance the ultrasound probe to visualize bladder space; 2) switch to sagittal view (using plane switch or button on the display screen to view the prostate; and 3) center the ultrasound probe from mid-prostate to bladder in the transverse view.

At a receive user input step 918, the user provides an input corresponding to one or more features of the user interface. In some embodiments, the input comprises an instruction to toggle a view from an image source such as an ultrasound probe as described herein. In some embodiments, the input comprises an instruction to proceed to a next stage, e.g. to one or more of a next mode, a next sub-mode, or a next step, for example.

In some embodiments, the user interface is configured to automatically proceed to the treatment probe insertion mode 920 in response to a user input indicating that the imaging probe insertion step has been completed, for example by the user selecting the next icon 334, e.g. the right arrow as described herein with reference to FIGS. 3A to 3F.

In some embodiments, the treatment probe insertion mode 920 comprises one or more of a user interface configuration step 922, a display primary and secondary images step 923, a display current mode step 924, a display instructions step 925, or a receive user input step 928.

At a user interface configuration step 922, one or more features of the user interface is displayed, such one or more of the user input features, the image display area, the primary image, the secondary image, the tool bar, the control panel, the displayed mode, the displayed sub-mode, the navigation bar as described herein, for example with reference to FIG. 3A. In some embodiments, the instructions executed by the one or more processors are configured to provide the one or more features of the configuration step 922 in response to a user input such as a user input selection indicating completion of a prior mode or sub-mode, e.g. the “next” icon of a prior mode or sub-mode such as mode or sub-mode 910.

At a display primary and secondary images step 923 of treatment probe insertion mode 920, the primary image comprises an endoscope image such as the cystoscope image and the secondary image comprises an ultrasound image such as an image from a TRUS probe. In some embodiments, the treatment probe comprises an endoscope such as a cystoscope as described herein. The real time ultrasound image may comprise a transverse image or a longitudinal image such as a sagittal image from the TRUS probe. In some embodiments, the real-time image from the TRUS probe shown in the secondary image display area and can be toggled between sagittal and transverse views in response to user input, for example. In some embodiments, user interface is configured to automatically display a real-time image from an endoscope in the image display area in the treatment probe insertion mode 920, e.g. in the primary image display area as described herein. In some embodiments, user interface is configured to automatically display a real-time image from the ultrasound probe in the secondary image display area in the treatment probe insertion mode 920, e.g. in the secondary image display area while the real-time image from the endoscope is displayed in the primary image display area.

At a display current mode step 924 of the treatment probe insertion mode 920, the user interface is configured to display the current setup mode 902 of the system and optionally the current treatment probe insertion sub-mode of the system, such as the treatment probe insertion mode 920, in which the treatment probe is inserted with a handpiece as described herein, for example with reference to FIG. 3A. In some embodiments, the displayed mode 902 is highlighted with respect to other displayed modes as described herein, e.g. plan mode 904, and treatment mode 906. In some embodiments, the current sub-mode 920 is displayed with the current mode 902 as described herein. In some embodiments, the current sub-mode 920 is highlighted with respect to other sub-modes 910, 930, that are also displayed with the current mode 902, for example with reference to FIG. 3A and the plurality of modes 340 and handpiece sub-mode 343, which corresponds to treatment probe insertion mode 920.

At a display instructions step 925, the user interface is configured to automatically display one or more instructions for the user to insert the treatment probe and optionally display the one or more instructions in a user input area of the control panel, for example as described herein with reference to FIG. 3A.

At a receive user input step 928, the user provides an input corresponding to one or more features of the user interface. In some embodiments, the input comprises an instruction to toggle a view from an image source such as an ultrasound probe as described herein. In some embodiments, the input comprises an instruction to proceed to a next stage, e.g. to one or more of a next mode, a next sub-mode, or a next step, for example for example by the user selecting the next icon 334, e.g. the right arrow, as described herein with reference to FIG. 3A.

In some embodiments, the probe alignment mode 930 comprises one or more of a user interface configuration step 932, a display primary and secondary images step 933, a display current mode step 934, a display instructions step 935, a display markers step 936, or a receive user input step 938. In some embodiments, the alignment mode 930 comprises a first configuration of the user interface at a first step of mode 930 to longitudinally align the treatment probe with the imaging probe, for example with reference to FIG. 3B, and a second configuration of the user interface at a second step of mode 930 to rotationally align the imaging probe with the treatment probe, for example with reference to FIG. 3C.

At a user interface configuration step 932, one or more features of the user interface is displayed, such as one or more of the user input features, the image display area, the primary image, the secondary image, the tool bar, the control panel, the displayed mode, the displayed sub-mode, the navigation bar as described herein, for example with reference to FIGS. 3B and 3C. In some embodiments, the instructions executed by the one or more processors are configured to provide the one or more features of the configuration step 932 in response to a user input such as a user input selection indicating completion of a prior mode or sub-mode, e.g. the “next” icon of a prior mode or sub-mode such as mode or sub-mode 920.

At a display primary and secondary images step 933, the user interface is configured to automatically display a real-time image from an ultrasound probe in the image display area in the alignment sub-mode 930. In some embodiments, the user interface is configured to automatically display the real time ultrasound image in the primary display area and the real time image from the endoscope such as a cystoscope in the secondary image display area, for example with reference to FIGS. 3B and 3C. In some embodiments, the user interface is configured for the user to toggle the real time ultrasound image between the longitudinal view, e.g. sagittal view, and the transverse view in response to a user input. Alternatively, the user interface can be configured to automatically switch from a longitudinal view, e.g. a sagittal view, to a transverse view in response to a user input such as an input to proceed to the next stage.

In some embodiments, the real time image that is automatically displayed comprises a longitudinal image in the primary image area, which can facilitate alignment of the longitudinal axis of the imaging probe with the treatment probe, for example by moving the imaging probe along the longitudinal axis of the imaging probe. In some embodiments, the real time image that is automatically displayed comprises a transverse image such as a transverse ultrasound image, for example. In some embodiments, the real time endoscope image is displayed as the secondary image in the alignment mode 930 as shown in FIGS. 3B and 3C, for example.

In some embodiments, user interface is configured to automatically display, in sequence, a longitudinal image and a transverse image in the alignment sub-mode. In some embodiments, the alignment mode 930 is configured to automatically display the longitudinal image first as the primary image to complete a first step of longitudinal alignment, as shown in FIG. 3B. In some embodiments, the user interface is configured to automatically display the transverse image as the primary image for the rotational alignment step as shown in FIG. 3C. In some embodiments, the transverse image is automatically displayed in response to a user input such as a next input, for example when a first step of the alignment mode 930 has been completed. Although reference is made to completing longitudinal alignment prior to rotational alignment, the order can be reversed, for example.

At a display current mode step 934, the user interface is configured to display the current set up mode 902 of the system and optionally the current alignment sub-mode 930 of the system, such as the alignment mode 930. In some embodiments, the displayed mode 902 is highlighted with respect to other displayed modes as described herein, e.g. plan mode 904, and treatment mode 906. In some embodiments, the current sub-mode 930 is displayed with the current mode 902 as described herein. In some embodiments, the current sub-mode 930 is highlighted with respect to other sub-modes 910, 920, that are displayed with the current mode 902, for example with reference to FIGS. 3B and 3C.

At a display user instructions step 935, user interface is configured to automatically display one or more alignment instructions to move one or more of the treatment probe or the ultrasound probe in the alignment sub-mode 930, and optionally to display the one or more instructions in a user input area of the control panel. In some embodiments, the one or more alignment instructions comprises an instruction to rotate an energy source of the probe about a longitudinal axis of the treatment probe to rotate an orientation of the energy source toward an elongate axis of the ultrasound probe in a transverse ultrasound image, for example.

In some embodiments, the one or more alignment instructions comprises a first instruction for the user to translate the imaging probe and a second instruction for the user to rotate one or more of the treatment probe or the imaging probe.

In some embodiments, the first user instruction comprises an instruction to translate the ultrasound probe along a longitudinal axis of the ultrasound probe to align a marker corresponding to an end of the ultrasound probe overlaid on a longitudinal ultrasound image with a position of an energy source of the treatment probe shown in the longitudinal image in the image display area, for example as shown in FIG. 3B. The first instruction may comprise an instruction to provide an input to proceed to a next step, such as by pressing a next icon as described herein.

In some embodiments, the second user instructions comprise an instruction to rotate the imaging probe such as a TRUS probe, so that the handpiece is located vertically above the imaging probe shown in a transverse image as shown in FIG. 3C. In some embodiments, the second instruction comprises an instruction to activate the energy source such as a water jet, and an instruction to rotate the energy source so that the direction of energy released from the energy source is pointed toward the imaging probe, for example to point vertically downward toward the imaging probe as shown in the transverse image of FIG. 3C.

At a display markers step 936, the user interface is configured to display one or more markers to aid in the alignment of the treatment probe and the imaging probe such as the TRUS probe. In some embodiments, the user interface is configured to display a flashing marker, such as a flashing colored marker, e.g. a flashing blue marker. In some embodiments, the marker is placed at a location corresponding to an end of the imaging probe. In some embodiments, the imaging probe is configured to translate along a longitudinal axis of the probe to bring the marker into alignment with the treatment probe. While the imaging probe can be translated in many ways, in some embodiments, a stage, such as stage with a stepper is moved with rotation of a knob as described herein. In some embodiments, the imaging probe is supported with a motorized stage, which allows the user to input one or more instructions to move the probe with the user interface, for example.

At a receive user input step 938, the user provides an input corresponding to one or more features of the user interface. In some embodiments, the input comprises an instruction to proceed to a next stage, e.g. to one or more of a next mode, a next sub-mode, or a next step, for example.

In some embodiments, upon completion of the setup mode 902, the user interface proceeds to the plan mode 904, which may comprise one or more of angle and depth mode 940, a registration mode 950, or a profile mode 960, for example.

In some embodiments, the angle and depth mode 940 comprises one or more of a user interface configuration step 942, a display primary and secondary images step 943, a display current mode step 944, a display instructions step 945, a display markers step 946, or a receive user input step 948.

At a user interface configuration step 942, one or more features of the user interface is displayed, such one or more of the user input features, the image display area, the primary image, the secondary image, the tool bar, the control panel, the displayed mode, the displayed sub-mode, the navigation bar as described herein, for example as described herein with reference to FIG. 3D. In some embodiments, the instructions executed by the one or more processors are configured to provide the one or more features of the configuration step 942 in response to a user input such as a user input selection indicating completion of a prior mode or sub-mode, e.g. the “next” icon of a prior mode or sub-mode such as mode or sub-mode 930. In some embodiments, the user interface comprises an input feature for a user to select an assisted planning mode. In some embodiments, the input is located in the control panel, for example.

Examples of tissue recognition algorithms suitable for use with assisted planning are described in PCT/US2019/038574, filed Jun. 21, 2019, entitled “ARTIFICIAL INTELLIGENCE FOR ROBOTIC SURGERY”, published as WO/2019/246580 on Jun. 26, 2019, and U.S. application Ser. No. 18/163,187, filed on Feb. 1, 2023, “USER INTERFACE FOR THREE DIMENSIONAL IMAGING AND TREATMENT”.

At a display primary and secondary images step 943 of the angle and depth mode 940, the user interface is configured to automatically display a real-time image from an imaging probe in the image display area, such as an image from an ultrasound probe, for example the TRUS probe. In some embodiments, the user interface is configured to automatically display a real time image from an ultrasound probe in the primary image display area and to automatically display the real time endoscope image such as the cystoscope in the secondary image display area, for example with reference to FIG. 3D. In some embodiments, the real time ultrasound image comprises a transverse image in a primary image display area and the real time endoscope image is displayed in the secondary image display area. In some embodiments, a transverse image is automatically displayed in the primary display area, for example a real time transverse image from TRUS probe as described herein.

In some embodiments, the user interface comprises a plurality of inputs such as tabs for a user to select a transverse image to display. In some embodiments, the user interface is configured to highlight a selected tab among the plurality tabs, in which each of the plurality of tabs corresponds to a selected transverse image.

In some embodiments, the user interface is configured to automatically display a transverse image among a plurality of transverse images. In some embodiments, the user interface is configured to automatically display a transverse image such as a transverse image of a median lobe of a prostate, for example. In some embodiments, the automatically generated image comprise a different image, such as a mid-prostate, if a “no median lobe” input has been selected by the user.

In some embodiments, the user interface is configured to automatically display another transverse image for planning in response to the user selecting the next button, which can allow the user to sequentially plan the treatment profile and adjust the treatment markers on each of the plurality of transverse images. In some embodiments, each of the plurality of displayed transverse images is overlaid with one or more treatment markers, which can be adjusted with user input as described herein, for example with reference to FIG. 3D.

At a display current mode step 944, the user interface is configured to display the current plan mode 904 of the system and optionally the current sub-mode 940 of the system, such as the angle and depth mode 940. In some embodiments, the displayed mode 904 is highlighted with respect to other displayed modes as described herein, e.g. set up mode 902, and treatment mode 906. In some embodiments, the current sub-mode 940 is displayed with the current mode 904 as described herein. In some embodiments, the current sub-mode 940 is highlighted with respect to other sub-modes, such as registration sub-mode 950 and the profile sub-mode 960 that are displayed with the current mode 904, for example with reference to FIG. 3D.

At a display instructions step 945, user interface is configured to automatically display one or more angle and depth instructions to move one or more treatment markers overlaid on an image, and optionally to display the one or more instructions in a user input area of the control panel, such as between the tool bar and marker select inputs as described herein. In some embodiments, the instructions comprise instructions to adjust one or more of an angle of a treatment plan or a depth of a treatment plan, for example. In some embodiments, the user interface is configured to provide one or more instructions to adjust the plurality of treatment markers to adjust one or more of an angle or a depth of treatment from the treatment probe, for example with reference to FIG. 3D.

At a display markers step 946, the user interface is configured to display one or more treatment markers, for example as shown with reference to FIG. 3D. In some embodiments, the user interface comprises one or more marker select inputs for a user to select a treatment marker to be adjusted as described herein. The angle and depth of a treatment marker can be adjusted by the user as described herein.

In some embodiments as part of the display markers step 946, the user interface is configured to display one or more treatment planning markers and an associated one or more treatment limit markers overlaid on one or more images, such as one or more transverse images. Although reference is made to one or more transverse images, the one or more treatment limit markers can be overlaid on longitudinal images as described herein. In some embodiments, the user interface is configured to provide feedback to the user in response to the one or more planning markers approaching the associated limit marker, the feedback comprising one or more of highlighting the marker, changing a color of the marker, flashing the marker, bracketing the marker, providing a pop-up window, or an auditory cue, for example. In some embodiments, the feedback can be provided to the selected treatment marker by limiting movement of the treatment marker beyond the limit marker. Alternatively or in combination, the treatment marker can be configured to bounce back to the limit marker when the treatment marker is moved by the user beyond the limit marker.

At a receive user input step 948, the user provides an input corresponding to one or more features of the user interface. The user input may comprise one or more of an input to select a treatment marker or to adjust one or more markers corresponding to an angle of treatment or a depth of treatment as shown on the one or more transverse images, for example as show with reference to FIG. 3D. In some embodiments, the input comprises an instruction to proceed to a next stage, e.g. to one or more of a next mode, a next sub-mode, or a next step, for example.

In some embodiments, the user interface proceeds to a registration mode 950 in response to the user selecting a next input.

In some embodiments, the registration mode 950 comprises one or more of a user interface configuration step 952, a display primary and secondary images step 953, a display current mode step 954, a display instructions step 955, a display markers step 956, or a receive user input step 958.

At user interface configuration step 952, one or more features of the user interface is displayed, such one or more of the user input features, the image display area, the primary image, the secondary image, the tool bar, the control panel, the displayed mode, the displayed sub-mode, the navigation bar as described herein, for example with reference to FIG. 3E. In some embodiments, the instructions executed by the one or more processors are configured to provide the one or more features of the configuration step 952 in response to a user input such as a user input selection indicating completion of a prior mode or sub-mode, e.g. the “next” icon of a prior mode or sub-mode such as mode or sub-mode 940. In some embodiments, the user interface comprises an input feature for a user to select an assisted planning mode. In some embodiments, the input is located in the control panel, for example.

At a display primary and secondary images step 953, the user interface is configured to automatically display a real-time image from a probe such as an ultrasound probe, for example the TRUS probe, in the image display area. In some embodiments, the user interface is configured to automatically display a real time image from an ultrasound probe in the primary image display area and to automatically display the real time endoscope image such as the cystoscope in the secondary image display area, for example with reference to FIG. 3E. In some embodiments, the automatically displayed real time ultrasound image comprises a longitudinal image in a primary image display area and the real time endoscope image is displayed in the secondary image display area. In some embodiments, a longitudinal image is automatically displayed in the primary display area, for example a real time transverse image from TRUS probe as described herein.

At a display current mode step 954, the user interface is configured to display the current plan mode 904 of the system and optionally the current sub-mode 950 of the system, such as the registration mode 940. In some embodiments, the displayed mode 904 is highlighted with respect to other displayed modes as described herein, e.g. set up mode 902, and treatment mode 906. In some embodiments, the current sub-mode 950 is displayed with the current mode 904 as described herein. In some embodiments, the current sub-mode 950 is highlighted with respect to other sub-modes, such as angle and depth sub-mode 940 and the profile sub-mode 960 that are displayed with the current mode 902.

At a display instructions step 955, the user interface is configured to automatically display one or more registration instructions for the user to move one or more registration markers overlaid on an image, and optionally to display the one or more instructions in a user input area of the control panel, such as between the tool bar and navigation bar. In some embodiments, in the registration mode, the user interface is configured to provide instructions to input a first location corresponding to a location of the energy source in the longitudinal image and to input a second location corresponding to a second location along a path of the energy source in the longitudinal image, for example with a first movable marker and a second movable marker. In some embodiments, the instructions comprise instructions to move the imaging probe such as the TRUS probe to see a desired anatomical tissue structure such as the bladder in a longitudinal image. In some embodiments, the instructions comprise instructions to place a first marker at a position corresponding to a position of the energy source such as a nozzle of a water jet, and a second marker at a position corresponding to a path of the energy source as described herein.

At a display markers step 956, the user interface is configured to display one or more markers, such a first marker showing a position of the energy source at a first location and second marker showing a path of the energy source at a second location. In some embodiments, the user interface is configured to display a user first user adjustable marker at the first location and a second user adjustable marker at a second location. In some embodiments, the user interface comprises one or more marker select inputs for a user to select the first marker or the second marker, which can be adjusted as described herein, e.g. with coarse or fine adjustment as described herein.

In some embodiments as part of the display markers step 956, the user interface is configured to display one or more treatment planning markers and an associated one or more treatment limit markers overlaid on one or more images, such as one or more longitudinal images. The associated one or more treatment limit markers may be displayed as a limit overlaid on the primary image, for example with reference to FIG. 3E. Although reference is made to one or more longitudinal images, the one or more treatment limit markers can be overlaid on transverse images as described herein.

In some embodiments, the associated one or more treatment limit markers is shown on a longitudinal image at a location corresponding to a maximum treatment depth of a corresponding transverse image. In some embodiments, the maximum treatment depth is located away from a plane of the longitudinal image on the corresponding transverse image in a transverse planning mode as described herein with reference to FIG. 3D, in which the second treatment marker (treatment marker 2) may be located at a greater distance from the probe than other markers such as the third and fourth treatment markers (treatment markers 3, 4, respectively).

In some embodiments, the user interface is configured to provide feedback to the user in response to the one or more planning markers approaching the associated limit marker, the feedback comprising one or more of highlighting the marker, changing a color of the marker, flashing the marker, bracketing the marker, providing a pop-up window, or an auditory cue, for example. In some embodiments, the feedback can be provided to the selected treatment marker by limiting movement of the treatment marker beyond the limit marker. Alternatively or in combination, the treatment marker can be configured to bounce back to the limit marker when the treatment marker is moved by the user beyond the limit marker.

At a receive user input step 958, the user provides an input corresponding to one or more features of the user interface. The user input may comprise an input to place one or more markers, or an input to adjust the one or more markers corresponding to a location of the energy source and a path of the energy source shown on the one or more longitudinal images. In some embodiments, the input comprises an instruction to proceed to a next stage, e.g. to one or more of a next mode, a next sub-mode, or a next step, for example.

In some embodiments, the user interface proceeds to a profile mode 960 in response to the user selecting a next input.

In some embodiments, the profile mode 960 comprises one or more of user interface configuration step 962, a display primary and secondary images step 963, a display current mode step 964, a display instructions step 965, a display markers step 966, or a receive user input step 968.

At a user interface configuration step 962, one or more features of the user interface is displayed, such one or more of the user input features, the image display area, the primary image, the secondary image, the tool bar, the control panel, the displayed mode, the displayed sub-mode, or the navigation bar as described herein, for example with reference to FIG. 3E. In some embodiments, the instructions executed by the one or more processors are configured to provide the one or more features of the configuration step 962 in response to a user input such as a user input selection indicating completion of a prior mode or sub-mode, e.g. the “next” icon of a prior mode or sub-mode such as mode or sub-mode 950.

At a display primary and secondary images step 963, the user interface is configured to automatically display a real-time image from a probe such as an ultrasound probe, for example the TRUS probe, in the image display area. In some embodiments, the user interface is configured to automatically display a real time image from an ultrasound probe in the primary image display area and to automatically display the real time endoscope image such as the cystoscope in the secondary image display area, for example with reference to FIG. 3E. In some embodiments, wherein the user interface is configured to automatically display a longitudinal ultrasound image in the profile sub-mode 960. In some embodiments, the automatically displayed real time image comprises a longitudinal image in a primary image display area and the real time endoscope image is displayed in the secondary image display area. In some embodiments, a longitudinal image is automatically displayed in the primary display area, for example a real time longitudinal image from TRUS probe as described herein.

At a display current mode step 964, the user interface is configured to display the current plan mode 904 of the system and optionally the current sub-mode 960 of the system, such as the profile mode 960. In some embodiments, the displayed mode 904 is highlighted with respect to other displayed modes as described herein, e.g. set up mode 902, and treatment mode 906. In some embodiments, the current sub-mode 960 is displayed with the current mode 904 as described herein. In some embodiments, the current sub-mode 960 is highlighted with respect to other sub-modes, such as angle and depth mode 940, registration sub-mode 950 that are displayed with the current mode 904.

At a display user instructions step 965, the user interface is configured to automatically display instructions to move one or more treatment markers overlaid on an image, and optionally to display the one or more instructions in a user input area of the control panel, such as between the tool bar and marker select inputs as described herein. In some embodiments, the user interface is configured to automatically provide one or more instructions to adjust the plurality of user adjustable treatment markers so as to adjust a profile of the treatment along the longitudinal image.

At a display markers step 966, the user interface is configured to display one or more treatment markers, for example as shown with reference to FIG. 3E. In some embodiments, in the profile sub-mode 960, the user interface is configured to provide one or more longitudinal images in the image display area with a plurality of user adjustable treatment markers for the user to plan the treatment. In some embodiments, a location of each of the plurality of user adjustable treatment markers shown on the longitudinal image corresponds to a location of a treatment profile of a corresponding transverse image.

In some embodiments, the user interface comprises one or more marker select inputs for a user to select a treatment marker to be adjusted as described herein. In some embodiments, the one or more markers comprise markers corresponding to a bladder neck marker or a veru protection zone as described herein, for example.

At a receive user input step 968, the user provides an input corresponding to one or more features of the user interface. The user input may comprise an input to adjust one or more markers as shown on the one or more longitudinal images, for example as show with reference to FIG. 3E. In some embodiments, the input comprises an instruction such as a user input selection to proceed to a next stage, e.g. to one or more of a next mode, a next sub-mode, or a next step, for example.

In some embodiments, the input comprises an input to accept the treatment plan, with an associated message and instructions. For example, the input to accept the treatment plan can be located adjacent a message to accept the treatment plan. In some embodiments, the input to accept the treatment plan and the corresponding message are located in a control panel as described herein.

In some embodiments, upon completion of the profile mode 904, the user interface proceeds to the treatment mode 906 in response to a user input such as a selection indicating completion of the profile mode. In some embodiments, the treatment mode 906 may comprises a treatment sub-mode 990, and a prime sub-mode 980 configured to prime the treatment probe after the treatment has started if appropriate, for example.

In some embodiments, the treatment mode or sub-mode 990 comprises one or more of a user interface configuration step 992, a display primary and secondary images step 993, a display current mode step 994, a display instructions step 995, a display markers step 996, or a receive input step 998.

At a user interface configuration step 992, one or more features of the user interface is displayed, such one or more of the user input features, the image display area, the primary image, the secondary image, the tool bar, the control panel, the displayed mode, the displayed sub-mode, the navigation bar as described herein, for example with reference to FIG. 3F. In some embodiments, the instructions executed by the one or more processors are configured to provide the one or more features of the configuration step 992 in response to a user input such as a user input selection indicating completion of a prior mode or sub-mode, e.g. the “next” icon of a prior mode or sub-mode such as mode or sub-mode 980. In some embodiments, a lock or other icon is displayed to indicate that the treatment plan has been accepted and locked subsequent to user acceptance.

In some embodiments, the user interface is configured to display one or more treatment parameters in the treatment mode, the one or more parameters comprising one or more of an elapsed treatment time, a treatment time remaining, a power level of the energy source, a sweep angle of the energy source, or an amount of progress of the treatment.

At a display primary and secondary images step 993, the user interface is configured to automatically display a real-time image from a probe such as an ultrasound probe, for example the TRUS probe, in the image display area. In some embodiments, the user interface is configured to automatically display a real time image from an ultrasound probe in the primary image display area and to automatically display the real time endoscope image such as the cystoscope in the secondary image display area, for example with reference to FIG. 3F. In some embodiments, the real time image comprises a longitudinal ultrasound image in a primary image display area and the real time endoscope image is displayed in the secondary image display area. In some embodiments, a longitudinal image is automatically displayed in the primary display area, for example a real time longitudinal image from TRUS probe as described herein.

At a display current mode step 994, the user interface is configured to display the current treat mode 906 of the system and optionally the current sub-mode 990 of the system, such as the treatment mode 990. In some embodiments, the displayed mode 906 is highlighted with respect to other displayed modes as described herein, e.g. set up mode 902, and plan mode 904. In some embodiments, the current sub-mode 990 is displayed with the current mode 906 as described herein. In some embodiments, the current sub-mode 990 is highlighted with respect to other sub-modes. Alternatively, the current sub-mode can be displayed without other sub-modes, for example as shown with reference to FIG. 3F.

At a display instructions step 995, the user interface is configured to automatically display instructions to provide one or more inputs related to the treatment of the patient, and optionally to display the one or more instructions in a user input area of the control panel, such as between the tool bar and the navigation as described herein with reference to FIG. 3F. The displayed instructions may comprise an instruction to press a foot pedal to begin the treatment, for example. The displayed instruction may comprise an instruction to lift the foot pedal to pause the treatment, for example.

At a display markers step 996, the user interface is configured to display one or more markers related to treatment, for example one or more markers as shown with reference to FIG. 3F. In some embodiments, one or more markers comprises a marker related to a longitudinal position of an energy source such as a water jet nozzle, for example.

At a receive input step 998, the user provides an input corresponding to one or more features of the user interface. The user input may comprise an input to adjust a position of an energy source or an amount of power from the energy source, such as a flow rate from a nozzle, for example. In some embodiments, one or more of the inputs can also be provided the handpiece as described herein.

In some embodiments, the user interface is configured to provide instructions to the user to prime the energy source, such as a water jet or a laser, for example. In some embodiments, the energy source is primed outside the patient and prior to insertion of the energy source into the patient. In some instances, a source of a fluid such as saline may not be sufficient to complete a treatment, and it can be helpful to prime the energy source such as a water jet a second time. In such situations, it can be helpful to leave the probe inside the patient when the energy source is primed a second time, for example so as to maintain longitudinal placement of the energy source when the treatment is paused.

In some embodiments, the user instructions are configured to display an instruction to prime an energy source prior to providing an instruction to insert a treatment probe comprising the energy source into a patient.

In some embodiments, the processor instructions are configured to reduce an amount of energy from the energy source if the system has entered a treatment mode or sub-mode and the energy source is primed while within a patient.

In some embodiments, the processor instructions are configured to automatically orient the energy source toward a portion of the probe if the system has entered a treatment mode or sub-mode and the energy source is primed while within the patient.

In some embodiments, the prime mode or sub-mode 980 comprises one or more of a user interface configuration step 982, a display primary and secondary images step 983, a display current mode step 984, a display instructions step 985, a display markers step 986, or a receive input step 988, which can be similar to the treatment sub-mode 990.

In some embodiments, the processor instructions are configured to automatically orient the energy source toward a portion of the probe if the system has entered a treatment mode or sub-mode and the user interface enters the prime mode or sub-mode 980 after the user interface has entered the treatment mode or sub-mode 990. In some embodiments, the processor instructions are configured to automatically decrease the amount of energy of the energy source to an amount less than the amount used during the treatment if the system has entered a treatment mode or sub-mode and the user interface enters the prime mode or sub-mode 980 after the user interface has entered the treatment mode or sub-mode 990.

At a user interface configuration step 982, one or more features of the user interface is displayed, such one or more of the user input features, the image display area, the primary image, the secondary image, the tool bar, the control panel, the displayed mode, the displayed sub-mode, the navigation bar as described herein, for example with reference to FIG. 3F. In some embodiments, the instructions executed by the one or more processors are configured to provide the one or more features of the configuration step 982 in response to a user input such as a user input selection indicating completion of a prior mode or sub-mode, e.g. the “next” icon as described herein. In some embodiments, a lock or other icon is displayed to indicate that the treatment plan has been accepted and locked while the energy source is primed.

In some embodiments, the user interface is configured to display one or more energy source priming parameters in the prime mode, the one or more parameters comprising one or more of a power level of the energy source, an angle of the energy source, or an amount of progress of the treatment when the treatment was paused, and priming mode initiated.

At a display primary and secondary images step 983, the user interface is configured to automatically display a real-time image from a probe such as an ultrasound probe, for example the TRUS probe, in the image display area. In some embodiments, the user interface is configured to automatically display a real time image from an ultrasound probe in the primary image display area and to automatically display the real time endoscope image such as the cystoscope in the secondary image display area, for example with reference to FIG. 3E. In some embodiments, the real time image comprises a longitudinal image in the primary image display area and the real time endoscope image is displayed in the secondary image display area. In some embodiments, a longitudinal image is automatically displayed in the primary display area, for example a real time longitudinal image from TRUS probe as described herein.

At a display current mode step 984, the user interface is configured to display the current treat mode 906 of the system and the current prime sub-mode 980 of the system. In some embodiments, the displayed mode 906 is highlighted with respect to other displayed modes as described herein, e.g. set up mode 902, and plan mode 904. In some embodiments, the current sub-mode 980 is displayed with the current treat mode 906 as described herein. In some embodiments, the current sub-mode 980 is highlighted with respect to other sub-modes, e.g. the treatment sub-mode 990. Alternatively, the current sub-mode can be displayed without other sub-modes, for example as shown with reference to FIG. 3F.

At a display instructions step 985, the user interface is configured to automatically display instructions to provide one or more inputs related to the treatment of the patient, and optionally to display the one or more instructions in a user input area of the control panel, such as between the tool bar and the navigation as described herein with reference to FIG. 3F. The displayed instructions may comprise an instruction to press a foot pedal to begin the priming of the energy source, for example. The displayed instruction may comprise an instruction to lift the foot pedal when priming of the energy source has been completed, for example.

At a display markers step 986, the user interface is configured to display one or more markers related to treatment, for example one or more markers as shown with reference to FIG. 3F. In some embodiments, one or more markers comprises a marker related to a longitudinal position of an energy source such as a water jet nozzle during the priming of the energy source, for example.

At a receive input step 988, the user provides an input corresponding to one or more features of the user interface. The user input may comprise an input to adjust a rotational angle of an energy source or an amount of power from the energy source, such as a flow rate from a nozzle, for example. In some embodiments, the one or more of the inputs can also be provided with the handpiece as described herein. In some embodiments, the user interface is configured for the user to adjust a rotational orientation of the energy source to direct energy from the energy source such as a water jet toward a portion of the probe, e.g. a stiff portion of the probe, to reduce likelihood of tissue being treated, e.g. resected, during the prime mode.

In some embodiments, the user interface is configured to automatically select an image source for an image to display in the image display area, the image source and the image corresponding to the current mode and a current sub-mode of the system. In some embodiments, this is performed each of a plurality of the display first and second images steps, such as two or more of steps 913, 923, 933, 943, 953, 963, 983, or 993, for example. In some embodiments, the user interface is configured to automatically select one or more of an endoscope image, an ultrasound image, a transverse ultrasound image or a longitudinal image to display in the image display area. In some embodiments, the user interface is configured to automatically display a primary image from a first image source and a secondary image from a secondary image source in the image display area. In some embodiments, the primary image source and the secondary image source correspond to the current mode and a current sub mode of the mode as described herein.

In some embodiments, the user interface is configured to automatically display a real time endoscope image from an endoscope in the primary image display area and a real time ultrasound image from an ultrasound device in the secondary image display area in a first mode, e.g. step 923 of treatment probe insertion mode 920, and to automatically display the real time ultrasound image in the primary image display area and the endoscope image in the secondary image display area in a second mode, for example with one or more of steps 933, 943, 953, 963, 983 or 993 of corresponding modes or sub-modes 930, 940, 950, 980 or 990, respectively, for example.

In some embodiments, the second mode the user interface is configured to automatically display a real time transverse ultrasound image in a first sub-mode 940 of the second mode and to automatically display a real time longitudinal ultrasound image in a second sub-mode 960 of the second mode, e.g. the plan mode 904.

In some embodiments, the user interface is configured to toggle between the real time transverse ultrasound image and the real time longitudinal ultrasound image in the first sub-mode and between the real time longitudinal ultrasound image and the real time transverse image in the second sub-mode of the second mode.

In some embodiments, the real time transverse ultrasound image and the real time longitudinal ultrasound image shown in the image display area remain partitioned from the control panel when toggled, e.g. above the control panel when toggled.

In some embodiments, wherein the user interface is configured to highlight a current mode of the plurality of modes, for example with reference one or more the display current mode steps 914, 924, 934, 944, 954, 964, 984 or 994.

In some embodiments, the user interface is configured to highlight each of the plurality of modes in a sequence. In some embodiments, the sequence corresponds to an order in which each of the modes and sub-modes are provided. In some embodiments each of the plurality of modes is highlighted in a predetermined sequence corresponding to a current mode of operation of the system. In some embodiments, the user interface is configured to sequentially highlight said each of the plurality of modes in a left to right order on the display, for example.

In some embodiments, an order the plurality of modes shown on the display remains fixed. In some embodiments, a location of each of the plurality of modes remains substantially fixed, for example at a location within a fixed area of the display. In some embodiments, each of the displayed modes 902, 904 and 906 can remain at the same location on the display while the highlighting changes to indicate which of the plurality of modes comprises the current mode, for example with reference to FIGS. 3A to 3F. In some embodiments, the user interface is configured to display the plurality of modes on a navigation bar, for example. In some embodiments, the plurality of modes is displayed between a previous icon and a next icon of the navigation bar.

In some embodiments, the user interface is configured to display the plurality of modes at a fixed area of the display, such as a fixed area between a previous icon and a next icon of a user interface. In some embodiments, the fixed area comprises a first fixed area to display the plurality of modes such as modes 902, 904 and 906, and a second fixed area to display a plurality of sub-modes adjacent the first fixed area, such as sub-modes 910, 920, 930, 940, 950, 960, 980 and 990, for example. In some embodiments, the interface is configured to display one or more sub-modes of the plurality of sub-modes at the second fixed area of the display when the displayed one or more sub-modes is associated with the current mode highlighted in the fixed area.

In some embodiments, each the plurality of modes 902, 904, 906 comprises one or more sub-modes, such as two or more of sub-modes 910, 920, 930, 940, 950, 960, 980 or 990. In some embodiments, each of the one or more sub-modes is shown on the display when the one or more sub-modes is associated with the current mode highlighted on the display, for example with reference to FIGS. 3A to 3F. In some embodiments, the one or more sub-modes shown on the display is highlighted with the current mode highlighted on the display. In some embodiments, the one or more sub-modes comprises a plurality of sub-modes associated with the current mode highlighted on the display. In some embodiments, the plurality of sub-modes associated with the current highlighted mode shown on the display is arranged in a sequence and sequentially highlighted. In some embodiments, the plurality of sub-modes is sequentially highlighted in a left to right sequence, for example with reference to FIGS. 3A to 3C and FIGS. 3D and 3E. In some embodiments, the one or more sub-modes comprises a plurality of sub-modes located on a navigation bar adjacent the plurality of modes. In some embodiments, the one or more sub-modes comprises a plurality of sub-modes located between a previous icon and a next icon.

In some embodiments, the user interface is configured to automatically select the primary image source displayed in the primary image display area and the secondary image source displayed in the secondary image display area so as to correspond with a current mode or sub-mode of the user interface. This automatic selection of the primary image source and the secondary image source can make it easier for the user to proceed through the modes and sub-modes and can improve safety of the procedure. In some embodiments, the primary image source comprises the ultrasound image source such as an ultrasound probe and the secondary image source comprises the endoscope image source such as an endoscope on a treatment probe, which can improve safety of the procedure because the user can view the endoscope image in the secondary display area while using the ultrasound image as the primary image, for example while aligning the probes, planning the angle and depth, registration of the treatment profile and probe, planning the treatment profile and treating the patient as described herein.

In some embodiments, the instructions executed by the one or more processors are configured to select in sequence: 1) an ultrasound image source as the primary image source for the primary image display area in a first mode or sub-mode to insert an ultrasound probe into a patient, such as during imaging probe insertion mode 910; 2) an endoscope image source as the primary image source for the primary display area and the ultrasound image as the secondary image source for the secondary image display area in a second mode or sub-mode to insert a treatment probe comprising an endoscope into the patient, such as treatment probe insertion mode 920; and 3) the ultrasound image source as the primary image source for the primary image display area and the endoscope image source as the secondary image source for the secondary image display area for each of a plurality of subsequent modes or sub-modes, such as two or more of probe alignment mode or sub-mode 930, angle and depth mode or sub-mode 940, registration mode or sub-mode 950, profile mode or sub-mode 960, prime mode or sub-mode 980 or treatment mode or sub-mode 990. In some embodiments, the plurality of subsequent modes or sub-modes comprises a planning mode or sub-mode 904 and a treatment mode or sub-mode 906, for example. In some embodiments, the plurality of subsequent modes or sub-modes comprises three or more of a probe alignment mode or sub-mode 930, an angle and depth mode or sub-mode 940, a registration mode or sub-mode 950, a profile mode or sub-mode 960, a prime mode 980, or a treatment mode or sub-mode, 990, for example.

In some embodiments, the user interface is configured to select an ultrasound image source as the primary image source in a mode corresponding to insertion of an ultrasound probe into the patient, e.g. mode 910, and an endoscope image source for the primary image display area in a mode corresponding to insertion of a surgical treatment probe into the patient, e.g. mode 920. In some embodiments, the processor is configured to select the ultrasound probe as the primary image source and the endoscope as the secondary image source for a plurality of modes subsequent to the mode corresponding to insertion of the treatment probe into the patient, such as two or more of probe alignment mode or sub-mode 930, angle and depth mode or sub-mode 940, registration mode or sub-mode 950, profile mode or sub-mode 960, prime mode or sub-mode 980 or treatment mode or sub-mode 990. In some embodiments, the plurality of modes subsequent to the mode of inserting the treatment probe comprises a planning mode 904, and a treatment mode 906, for example. In some embodiments, the plurality of modes subsequent to the mode of inserting the treatment probe comprises two or more of an alignment mode or sub-mode 930, an angle and depth mode or sub-mode 940, a registration mode or sub-mode 940, a profile mode or sub-mode 960, a prime mode or sub-mode 980, or a treatment mode or sub-mode 990. In some embodiments, the plurality of modes subsequent to inserting the treatment probe comprises at least four of the modes or sub-modes in which the primary image source comprises the ultrasound probe and the secondary image source comprises the endoscope.

Although FIG. 9 shows a user interface method 900 in accordance with some embodiments, one of ordinary skill in the art will recognize many adaptations and variations. For example, the steps, modes and sub-modes can be performed in a sequence as shown, or in a different order. Some of the steps, modes and sub-modes can be omitted and some of the steps repeated, for example.

Although the present disclosure makes reference to modes and sub-modes, any of the sub-modes described herein may comprise a mode, which can be performed independently of other modes and sub modes.

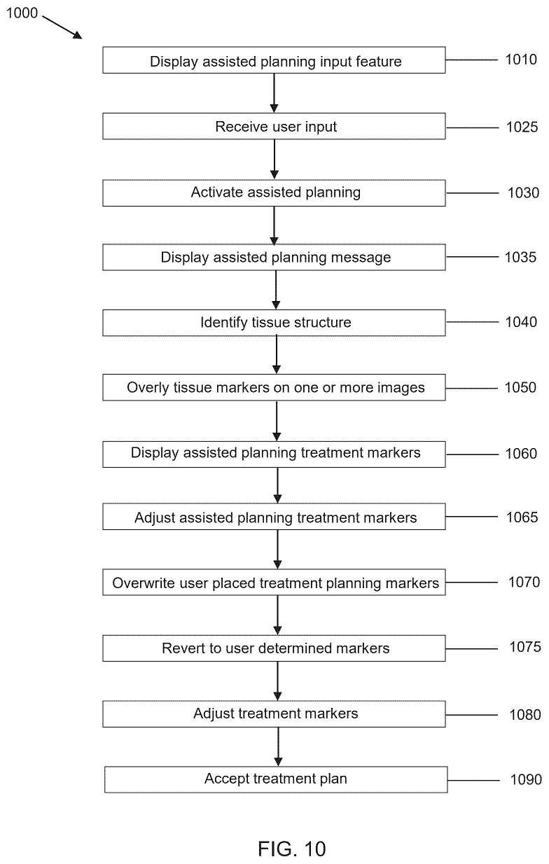

FIG. 10 shows a method 1000 of assisted tissue planning. The method 1000 can be combined with the user interface and method 900 as described herein.

At a step 1010, an assisted planning user input feature is displayed. The user input feature may comprise any suitable user input as described herein. In some embodiments, the input feature comprises one or more of a label, an icon, a toggle, a button, or a slider configured to receive the user input. In some embodiments, the user input comprises a toggle, for example as described with reference to FIGS. 3D and 3D. While the input feature to turn on assisted planning can be located anywhere on the display, in some embodiments the input is located on the control panel.

In some embodiments, the user interface is configured to present the assisted planning input in each of a plurality of transverse views to allow the user to select assisted planning in said each of the plurality of transverse views.

At a step 1025, a user input is received to activate assisted planning. In some embodiments, the input is configured to default to a setting from a previously completed treatment from a last treatment session of the system. For example, the input can default to assisted planning turned on if assisted planning was used for the last treatment session, or to default to off if assisted planning was not used during the last treatment session.

At a step 1030, assisted planning is activated in response to the user input. The activation of the assisted planning can trigger an image recognition process to be activated, such as an artificial intelligence algorithm configured to identify tissue structures from an image, such as a neural network, a convolutional neural network, or a machine learning algorithm, for example. Examples of suitable image recognition processes are described in PCT/US2019/038574, filed Jun. 21, 2019, entitled “ARTIFICIAL INTELLIGENCE FOR ROBOTIC SURGERY”, published as WO/2019/246580 on Jun. 26, 2019, and U.S. application Ser. No. 18/163,187, filed on Feb. 1, 2023, “USER INTERFACE FOR THREE DIMENSIONAL IMAGING AND TREATMENT”, the entire disclosures of which have been previously incorporated herein by reference.

At a step 1035, an assisted planning message is activated. The message may comprise any message indicated that an assisted planning process has been activated, such as a message that image recognition or tissue recognition is in process, for example. In some embodiments, a user input is provided to cancel assisted planning.

At a step 1040, a tissue structure is identified. The identified tissue structure may comprise any suitable tissue structure, such as a tissue boundary, a boundary of an organ such as the prostate, a capsule of an organ, a capsule of a prostate, a verumontanum of a prostate, a mid-prostate, a median lobe of a prostate, or a bladder neck, for example. In some embodiments in the assisted planning mode, the instructions of an artificial intelligence (AI) algorithm are configured to identify one or more anatomical tissue structures from one or more of a longitudinal image or a transverse image and to mark a location of the one or more tissue structure on the one or more of the longitudinal image or the transverse image.

In some embodiments, an alert is provided to the user if the boundary is not identified from the processed image, such as a transverse image or a longitudinal image, for example.

At a step 1050, at a step one or more tissue markers is overlaid on one or more images. The one or more markers may comprise a marker along a boundary of a tissue, such as a boundary of a prostate capsule, for example. In some embodiments, the marker comprises a color, change in contrast or other indicia such as a line to indicate the tissue structure. In some embodiments, the user interface is configured to mark a boundary of a tissue in a transverse image, for example.

At a step 1060, one or more assisted planning treatment markers is overlaid on one or more images. The assisted planning markers may comprise treatment markers overlaid on one or more transverse or longitudinal images, for example with reference treatment to FIGS. 3D and 3E. In some embodiments, processor instructions are configured to determine a treatment start location and a treatment end location and display a treatment start marker and a treatment end marker on the longitudinal image, for example as described herein with reference to FIG. 3E.

In some embodiments, the user interface is configured to display a plurality of treatment markers of a treatment plan overlaid on the image in response to a tissue structure such as a boundary. In some embodiments, the user interface is configured to display a treatment profile with the treatment markers, for example. In some embodiments, the treatment profile is determined in response to the tissue boundary.

In some embodiments, the processor instructions of the tissue recognition algorithm are configured to determine a plurality of locations of a plurality of treatment markers and display the plurality of treatment markers at the plurality of locations of a longitudinal image.

At a step 1065, one or more assisted planning treatment markers is adjusted. The one or more assisted planning treatment markers can be adjusted as described herein, for example with reference to FIGS. 3D and 3E. In some embodiments, in the assisted planning mode, the user interface is configured for the user to adjust the plurality of markers of the treatment plan overlaid on the image, for example.

At a step 1070, one or more treatment planning markers placed by the user is overwritten. In some embodiments, a user may start to plan a treatment and then decide to use assisted planning part way through the planning process. The treatment markers placed by the user can be overwritten with the assisted planning markers in response to the user selecting assisted planning. In some embodiments, the locations of the assisted planning markers placed by the user are overwritten with new locations determined with the assisted planning software. In some embodiments, the user interface is configured to overwrite one or more treatment planning markers overlaid on an image at one or more locations in response to a first user input with a plurality of assisted planning treatment markers in response to a second user input to select assisted planning after the user has positioned the one or more treatment planning markers.

At a step 1075, one or more displayed treatment markers reverts to user determined markers. In some embodiments, the user interface comprises an input to undo the assisted planning markers and revert to the one or more markers overlaid on the image at the one or more locations in response to the first user input. In some embodiments, the user may not be satisfied with the results of the assisted planning. In response to the user cancelling or otherwise turning off the assisted planning, for example with an input to a toggle, the one or more displayed treatment markers reverts to user determined markers. In some embodiments, the locations of the treatment markers are reverted to the locations of the user placed treatment markers. For example, the markers may comprise the same markers and only the locations of the markers reverts back to the user defined locations.

At a step 1080, one or more treatment markers is adjusted with user input as described herein.

At a step 1090, the treatment plan is accepted by the user. In some embodiments the treatment plan is accepted with an acknowledgement that assisted planning was used to plan the treatment. In some embodiments, the user interface is configured to display a notification that assisted planning was used with the input to accept the treatment plan upon completion of a planning mode 904 and prior to the treatment mode 906 as described with reference to FIG. 9.

Although FIG. 10 shows the method 1000 of assisted planning in accordance with some embodiments, one of ordinary skill in the art will recognize many adaptations and variations. For example, the steps, modes and sub-modes can be performed in a sequence as shown, or in a different order. Some of the steps, modes and sub-modes can be omitted and some of the steps repeated, for example.

Any mode, sub-mode or step of any method or user interface disclosed herein can be combined with any mode, sub-mode or step of any other method as disclosed herein.

A processor can be configured to perform the step of any method or process disclosed herein. One of ordinary skill in the art will recognize that a processor can be configured with instructions to perform one or more of modes, sub-modes, steps of any method or process as disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The “user interface” (UI) as described herein may comprise one or more components of UI as will be understood by one of ordinary skill in the art in computer science. The UI may comprise one or more components of a graphical user interface (GUI), and the GUI may comprise one or more input devices such as a display and one or more pointing devices such as touch pads, directional pads, push buttons, track pads, mice, joy sticks configured for a user to input commands to the computing device in association with the display. In some embodiments, the display comprises a touch screen display configured for the user to provide touch inputs to the computing device with the display. Alternatively or in combination, the UI may comprise voice commands or gesture sensing to allow the user to provide input such as input commands to the computing device. The processor can be configured to provide one or more features such as icons or controls on a display and to receive user input associated with the one or more features such as icons as described herein.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any process or method as disclosed herein can be combined with any one or more steps of any other process or method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

As used herein, the term “e.g.” means for example.

The present disclosure includes the following numbered clauses.

Clause 1. A user interface to operate a robotic system to perform a surgery, the user interface comprising: instructions stored on a computer readable medium, which when executed by one or more processors, cause the one or more processors to: provide a plurality of images on an image display area of a touchscreen display; provide a control panel on the touchscreen display, the control panel partitioned from the image display area, the control panel comprising a plurality of features to receive a plurality of user inputs in response to the user touching the plurality of features; display a current mode of a plurality of modes of the system.

Clause 2. The user interface of any of the preceding clauses, wherein the control panel is located below the image display area.

Clause 3. The user interface of any of the preceding clauses, wherein the instructions are configured to select in sequence: 1) an ultrasound image source as the primary image source for the primary image display area in a first mode or sub-mode to insert an ultrasound probe into a patient; 2) an endoscope image source as the primary image source for the primary display area and the ultrasound image as the secondary image source for the secondary image display area in a second mode or sub-mode to insert a treatment probe comprising an endoscope into the patient; and 3) the ultrasound image source as the primary image source for the primary image display area and the endoscope image source as the secondary image source for the secondary image display area for each of a plurality of subsequent modes or sub-modes.

Clause 4. The user interface of any of the preceding clauses, wherein the plurality of subsequent modes or sub-modes comprises a planning mode or sub-mode and a treatment mode or sub-mode.

Clause 5. The user interface of any of the preceding clauses, wherein the plurality of subsequent modes or sub-modes comprises three or more of a probe alignment mode or sub-mode, an angle and depth mode or sub-mode, a registration mode or sub-mode, a profile mode or sub-mode, or a treatment mode or sub-mode.

Clause 6. The user interface of any of the preceding clauses, wherein the user interface is configured to automatically select an image source for an image to display in the image display area, the image source and the image corresponding to a current mode and a current sub-mode of the system.

Clause 7. The user interface of any of the preceding clauses, wherein the instructions are configured to select a primary image source for a primary image display area and a secondary image source corresponding to a secondary image display area in response to the current mode and the current sub-mode of the system.

Clause 8. The user interface of any of the preceding clauses, wherein the instructions are configured to select an ultrasound image source as the primary image source in a mode corresponding to insertion of an ultrasound probe into the patient and an endoscope image source for the primary image display area in a mode corresponding to insertion of a surgical treatment probe into the patient.

Clause 9. The user interface of any of the preceding clauses, wherein the processor is configured to select the ultrasound probe as the primary image source and the endoscope image source as the secondary image source for a plurality of modes subsequent to the mode corresponding to insertion of the treatment probe into the patient.

Clause 10. The user interface of any of the preceding clauses, wherein the plurality of modes subsequent to the mode of inserting the treatment probe comprises a planning mode and a treatment mode.

Clause 11. The user interface of any of the preceding clauses, wherein the plurality of modes subsequent to the mode of inserting the treatment probe comprises two or more of an alignment mode, an angle and depth mode, a registration mode, a profile mode or a treatment mode.

Clause 12. The user interface of any of the preceding clauses, wherein the plurality of modes subsequent to inserting the treatment probe comprises at least four modes in which the primary image source comprises the ultrasound probe and the secondary image source comprises the endoscope.

Clause 13. The user interface of any of the preceding clauses, wherein the user interface is configured to automatically select one or more of an endoscope image, an ultrasound image, a transverse ultrasound image or a longitudinal image to display in the image display area.

Clause 14. The user interface of any of the preceding clauses, wherein the user interface is configured to display a primary image from a first image source and a secondary image from a secondary image source in the image display area.

Clause 15. The user interface of any of the preceding clauses, wherein the primary image source and the secondary image source correspond to the current mode and the current sub mode of the mode.

Clause 16. The user interface of any of the preceding clauses, wherein the user interface is configured to automatically display a real time endoscope image from an endoscope in the primary image display area and a real time ultrasound image from an ultrasound device in the secondary image display area in a first mode and to automatically display the real time ultrasound image in the primary image display area and the endoscope image in the secondary image display area in a second mode.

Clause 17. The user interface of any of the preceding clauses, wherein in the second mode the user interface is configured to automatically display a real time transverse ultrasound image in a first sub-mode of the second mode and to automatically display a real time longitudinal ultrasound image in a second sub-mode of the second mode.

Clause 18. The user interface of any of the preceding clauses, wherein the user interface is configured to toggle between the real time transverse ultrasound image and the real time longitudinal ultrasound image in the first sub-mode of the second mode and between the real time longitudinal ultrasound image and the real time transverse image in the second sub-mode of the second mode.

Clause 19. The user interface of any of the preceding clauses, wherein the real time transverse ultrasound image and the real time longitudinal ultrasound image shown in the image display area remain above the control panel when toggled.

Clause 20. The user interface of any of the preceding clauses, wherein the user interface is configured to highlight a current mode of the plurality of modes.

Clause 21. The user interface of any of the preceding clauses, wherein the user interface is configured to highlight each of the plurality of modes in a sequence.

Clause 22. The user interface of any of the preceding clauses, wherein the said each of the plurality of modes is highlighted in a predetermined sequence corresponding to a current mode of operation of the system.

Clause 23. The user interface of any of the preceding clauses, wherein the user interface is configured to sequentially highlight said each of the plurality of modes in a left to right order on the display.

Clause 24. The user interface of any of the preceding clauses, wherein an order the plurality of modes shown on the display remains fixed.

Clause 25. The user interface of any of the preceding clauses, wherein a location of each of the plurality of modes remains substantially fixed.

Clause 26. The user interface of any of the preceding clauses, wherein the user interface is configured to display the plurality of modes on a navigation bar.

Clause 27. The user interface of any of the preceding clauses, wherein the plurality of modes is displayed between a previous icon and a next icon of the navigation bar.

Clause 28. The user interface of any of the preceding clauses, wherein the user interface is configured to display the plurality of modes at a fixed area of the display.

Clause 29. The user interface of any of the preceding clauses, wherein the fixed area comprises a first fixed area to display the plurality of modes and a second fixed area to display a plurality of sub-modes adjacent the first fixed area.

Clause 30. The user interface of any of the preceding clauses, wherein the user interface is configured to display one or more sub-modes of the plurality of sub-modes at the second fixed area of the display when the displayed one or more sub-modes is associated with the current mode highlighted in the fixed area.

Clause 31. The user interface of any of the preceding clauses, wherein each the plurality of modes comprises one or more sub-modes.

Clause 32. The user interface of any of the preceding clauses, wherein each of the one or more sub-modes is shown on the display when the one or more sub-modes is associated with the current mode highlighted on the display.

Clause 33. The user interface of any of the preceding clauses, wherein the one or more sub-modes shown on the display is highlighted with the current mode highlighted on the display.

Clause 34. The user interface of any of the preceding clauses, wherein the one or more sub-modes comprises a plurality of sub-modes associated with the current mode highlighted on the display.

Clause 35. The user interface of any of the preceding clauses, wherein the plurality of sub-modes associated with the current highlighted mode shown on the display is arranged in a sequence and sequentially highlighted.

Clause 36. The user interface of any of the preceding clauses, wherein the plurality of sub-modes is sequentially highlighted in a left to right sequence.

Clause 37. The user interface of any of the preceding clauses, wherein the one or more sub-modes comprises a plurality of sub-modes located on a navigation bar adjacent the plurality of modes.

Clause 38. The user interface of any of the preceding clauses, wherein the one or more sub-modes comprises a plurality of sub-modes located between a previous icon and a next icon.

Clause 39. The user interface of any of the preceding clauses, wherein the plurality of modes comprises one or more of a setup mode to set up the system, a plan mode to plan a treatment with the system, or a treat mode to treat the patient with the system.

Clause 40. The user interface of c any of the preceding clauses, wherein the setup mode comprises one or more of an ultrasound probe insertion sub-mode, a treatment probe insertion sub-mode or an alignment sub-mode.

Clause 41. The user interface of any of the preceding clauses, wherein user interface is configured to automatically display a real-time image from the ultrasound probe in the image display area in the ultrasound probe insertion sub-mode.

Clause 42. The user interface of any of the preceding clauses, wherein user interface is configured to automatically display one or more instructions to insert the ultrasound treatment probe in the ultrasound insertion sub-mode and optionally display the one or more instructions in a user input area of the control panel.

Clause 43. The user interface of any of the preceding clauses, wherein user interface is configured to automatically display a real-time image from an endoscope in the image display area in the treatment probe insertion sub-mode.

Clause 44. The user interface of any of the preceding clauses, wherein user interface is configured to automatically display one or more instructions to insert the treatment probe in the treatment probe insertion sub-mode and optionally display the one or more instructions in a user input area of the control panel.

Clause 45. The user interface of any of the preceding clauses, wherein user interface is configured to automatically display a real-time image from an ultrasound probe in the image display area in the alignment sub-mode.

Clause 46. The user interface of any of the preceding clauses, wherein user interface is configured to automatically display, in sequence, a longitudinal image and a transverse image in the alignment sub-mode.

Clause 47. The user interface of any of the preceding clauses, wherein user interface is configured to automatically display one or more alignment instructions to move one or more of the treatment probe or the ultrasound probe in the alignment sub-mode and optionally display the one or more instructions in a user input area of the control panel.

Clause 48. The user interface of any of the preceding clauses, wherein the one or more alignment instructions comprises an instruction to translate the ultrasound probe along a longitudinal axis of the ultrasound probe to align a marker corresponding to an end of the ultrasound probe overlaid on a longitudinal ultrasound image with a position of an energy source of the treatment probe shown in the longitudinal image in the image display area.

Clause 49. The user interface of any of the preceding clauses, wherein the user interface is configured to automatically display the longitudinal image in the image display area.

Clause 50. The user interface of any of the preceding clauses, wherein the one or more alignment instructions comprises an instruction to rotate the ultrasound probe about a longitudinal axis of the ultrasound probe so as to rotate a position of a treatment probe in a transverse ultrasound image.

Clause 51. The user interface of any of the preceding clauses, wherein the user interface is configured to automatically display the transverse ultrasound image in the image display area.

Clause 52. The user interface of any of the preceding clauses, wherein the one or more alignment instructions comprises an instruction to rotate an energy source of the probe about a longitudinal axis of the treatment probe to rotate an orientation of the energy source toward an elongate axis of the ultrasound probe in a transverse ultrasound image.

Clause 53. The user interface of any of the preceding clauses, wherein the user interface is configured to automatically display the transverse ultrasound image in the image display area.

Clause 54. The user interface of any of the preceding clauses, wherein the plan mode comprises one or more of an angle and depth sub-mode, a registration sub-mode, or a profile sub-mode.

Clause 55. The user interface of any of the preceding clauses, wherein the user interface is configured to automatically display a transverse image in the angle and depth sub-mode.

Clause 56. The user interface of any of the preceding clauses, wherein in angle and depth sub-mode the user interface is configured to provide a plurality of transverse images in the image display area with a plurality of treatment markers for the user to plan the treatment.

Clause 57. The user interface of any of the preceding clauses, wherein the user interface comprises a plurality of tabs for a user to select an image among the plurality of transverse images, the user interface configured to show the plurality of treatment markers on each of the plurality of transverse images for the user to plan the treatment.

Clause 58. The user interface of any of the preceding clauses, wherein the user interface is configured to highlight a selected tab among the plurality tabs.

Clause 59. The user interface of any of the preceding clauses, wherein the user interface is configured to provide one or more instructions to adjust the plurality of treatment markers to adjust one or more of an angle or a depth of treatment from the treatment probe.

Clause 60. The user interface of any of the preceding clauses, wherein the user interface is configured to automatically display a longitudinal image in the registration sub-mode.

Clause 61. The user interface of any of the preceding clauses, wherein, in the registration sub-mode, the user interface is configured to provide instructions to input a first location corresponding to a location of the energy source in the longitudinal image and to input a second location corresponding to a second location along a path of the energy source in the longitudinal image.

Clause 62. The user interface of any of the preceding clauses, wherein the user interface is configured to display a user first user adjustable marker at the first location and a second user adjustable marker at a second location.

Clause 63. The user interface of any of the preceding clauses, wherein the user interface is configured to automatically display a longitudinal image in the profile sub-mode.

Clause 64. The user interface of any of the preceding clauses, wherein in the profile sub-mode, the user interface is configured to provide one or more longitudinal images in the image display area with a plurality of user adjustable treatment markers for the user to plan the treatment.

Clause 65. The user interface of any of the preceding clauses, wherein a location of each of the plurality of user adjustable treatment markers shown on the longitudinal image corresponds to a location of a treatment profile of a corresponding transverse image.

Clause 66. The user interface of any of the preceding clauses, wherein the user interface is configured to provide one or more instructions to adjust the plurality of user adjustable treatment markers so as to adjust a profile of the treatment along the longitudinal image.

Clause 67. The user interface of any of the preceding clauses, wherein the user interface is configured to display an input and an instruction to accept the treatment plan after completion of the planning mode.

Clause 68. The user interface of any of the preceding clauses, wherein the treat mode comprises a treatment sub-mode.

Clause 69. The user interface of any of the preceding clauses, wherein the user interface is configured to highlight the treatment mode and the treatment sub-mode on the display.

Clause 70. The user interface of any of the preceding clauses, wherein the user interface is configured to display one or more treatment parameters in the treatment mode, the one or more parameters comprising one or more of an elapsed treatment time, a treatment time remaining, a power level of the energy source, a sweep angle of the energy source, or an amount of progress of the treatment.

Clause 71. The user interface of any of the preceding clauses, wherein the user interface is configured for the user to touch the touchscreen display a total number of times at a plurality of locations to set up the treatment, plan the treatment and perform the treatment, and wherein at least half of the plurality of locations touched the total number of times is located below a centerline of the display.

Clause 72. The user interface of any of the preceding clauses, wherein the plurality of features is configured to receive the plurality of user inputs to plan a treatment of the patient with the plurality of user inputs located below the image display area.

Clause 73. The user interface of any of the preceding clauses, wherein an input to toggle between a transverse ultrasound image and a longitudinal ultrasound image is located below the image display area.

Clause 74. The user interface of any of the preceding clauses, wherein the control panel is configured to display one or more user instructions associated with the current mode.

Clause 75. The user interface of any of the preceding clauses, wherein the user interface is configured to display one or more user instructions associated with a current sub-mode of the current mode for each of the plurality of modes in the control panel below the image display area.

Clause 76. The user interface of any of the preceding clauses, wherein the user interface is configured to display the one or more instructions on the control panel.

Clause 77. The user interface of any of the preceding clauses, wherein the control panel comprises a feature for a user to view an instructional video associated with a current sub-mode of the current highlighted mode for each of the plurality of modes.

Clause 78. The user interface of any of the preceding clauses, wherein the control panel comprises a tool bar configured for a user to one or more of toggle an ultrasound image between a longitudinal view and a transverse view, adjust image settings of one or more imaging devices, adjust a longitudinal position of an energy source, display a measurement scale, or display system information.

Clause 79. The user interface of any of the preceding clauses, wherein the control panel comprises a plurality of marker select features for a user to select a treatment marker to adjust among a plurality of markers overlaid on an image in the image display area.

Clause 80. The user interface of any of the preceding clauses, wherein the plurality of treatment features selectable with the plurality of marker select buttons comprises one or more of a treatment start icon to select a starting location of treatment, a treatment end icon to select and ending location of the treatment, and a plurality of treatment icons overlaid on the image between the treatment start icon and the treatment stop icon.

Clause 81. The user interface of any of the preceding clauses, wherein the use interface is configured to provide a coarse adjustment to the selected treatment marker with a user touching the selected marker and sliding the selected marker from a first location on the image to a second location on the image.

Clause 82. The user interface of any of the preceding clauses, wherein the user interface is configured to display a directional pad comprising a plurality of directional icons in the control panel to adjust the selected treatment marker with user input to the directional pad.

Clause 83. The user interface of any of the preceding clauses, wherein the user interface is configured to constrain movement of the selected marker and to highlight one or more of the directional icons relative to other directional icons of the plurality of directional icons, the highlighted one or more of the directional icons corresponding to allowed movement of the marker.

Clause 84. The user interface of any of the preceding clauses, wherein the user interface is configured to display one or more treatment planning markers and an associated one or more treatment limit markers overlaid on one or more images.

Clause 85. The user interface of any of the preceding clauses, wherein the user interface is configured to provide feedback to the user in response to the one or more planning markers approaching the associated limit marker, the feedback comprising one or more of highlighting the marker, changing a color of the marker, flashing the marker, bracketing the marker, providing a pop-up window, or an auditory cue.

Clause 86. The user interface of any of the preceding clauses, wherein the user interface is configured to display the one or more treatment markers and the associated one or more treatment limit markers on one or more of a longitudinal image or a transverse image.

Clause 87. The user interface of any of the preceding clauses, wherein the associated one or more treatment limit markers is shown on a longitudinal image at a location corresponding to a maximum treatment depth of a corresponding transverse image.

Clause 88. The user interface of any of the preceding clauses, wherein the maximum treatment depth is located away from a plane of the longitudinal image on the corresponding transverse image in a transverse planning mode.

Clause 89. The user interface of any of the preceding clauses, wherein the user interface comprises an input feature for a user to select an assisted planning mode.

Clause 90. The user interface of any of the preceding clauses, wherein the input is located on the control panel.

Clause 91. The user interface of any of the preceding clauses, wherein the user interface is configured to display a notification that assisted planning was used with an input to accept the treatment plan upon completion of a planning mode and prior to a treatment mode.

Clause 92. The user interface of any of the preceding clauses, wherein the input is configured to default to a setting from a previously completed treatment from a last treatment session of the system.

Clause 93. The user interface of any of the preceding clauses, wherein the user interface is configured to overwrite one or more treatment planning markers overlaid on an image at one or more locations in response to a first user input with a plurality of assisted planning treatment markers in response to a second user input to select assisted planning after the user has positioned the one or more treatment planning markers.

Clause 94. The user interface of any of the preceding clauses, wherein the user interface comprises an input to undo the assisted planning markers and revert to the one or more markers overlaid on the image at the one or more locations in response to the first user input.

Clause 95. The user interface of any of the preceding clauses, wherein in an assisted planning mode, the user interface is configured to display a message that tissue recognition from an image is in progress.

Clause 96. The user interface of any of the preceding clauses, wherein the input feature comprises one or more of a label, an icon, a toggle, a button, or a slider configured to receive the user input.

Clause 97. The user interface of any of the preceding clauses, wherein in the assisted planning mode, the user interface is configured to identify one or more anatomical tissue structures from one or more of a longitudinal image or a transverse image and to mark a location of the one or more tissue structure on the one or more of the longitudinal image or the transverse image.

Clause 98. The user interface of any of the preceding clauses, wherein the one or more anatomical structures comprises one or more of a bladder neck, a mid-prostate, a median lobe of a prostate, a verumontanum of a prostate or a capsule of a prostate.

Clause 99. The user interface of any of the preceding clauses, wherein in the assisted planning mode, user interface is configured to determine a treatment start location and a treatment end location and display a treatment start marker and a treatment end marker on the longitudinal image.

Clause 100. The user interface of any of the preceding clauses, wherein in the assisted planning mode, the user interface is configured to mark a boundary of a tissue in a transverse image and optionally wherein the boundary comprises a boundary of an organ.

Clause 101. The user interface of any of the preceding clauses, wherein user interface is configured to provide an alert to the user if the boundary is not identified from the transverse image.

Clause 102. The user interface of any of the preceding clauses, wherein in the assisted planning mode, the user interface is configured to display a plurality of treatment markers of a treatment plan overlaid on the image in response to the boundary.

Clause 103. The user interface of any of the preceding clauses, wherein in the assisted planning mode, the user interface is configured for the user to adjust the plurality of markers of the treatment plan overlaid on the image.

Clause 104. The user interface of any of the preceding clauses, wherein the user interface is configured to present the assisted planning input in each of a plurality of transverse views to allow the user to select assisted planning in said each of the plurality of transverse views.

Clause 105. The user interface of any of the preceding clauses, wherein in the assisted planning mode, the user interface is configured to determine a plurality of locations of a plurality of treatment markers and display the plurality of treatment markers at the plurality of locations of a longitudinal image.

Clause 106. The user interface of any of the preceding clauses, wherein the user instructions are configured to display an instruction to prime an energy source prior to providing an instruction to insert a treatment probe comprising the energy source into a patient.

Clause 107. The user interface of any of the preceding clauses, wherein the processor instructions are configured to reduce an amount of energy from the energy source if the system has entered a treatment mode or sub-mode and the energy source is primed while within a patient.

Clause 108. The user interface of any of the preceding clauses, wherein the processor instructions are configured to orient the energy source toward a portion of the probe if the system has entered a treatment mode or sub-mode and the energy source is primed while within a patient.

Clause 109. The user interface of any of the preceding clauses, wherein the interface is configured to work with a first display and a second display, the first display configured to receive input from a surgeon while the second display substantially mirrors the first display.

Clause 110. The user interface of any of the preceding clauses, wherein the user interface is configured to allow a user at the second display to input annotations that are visible on the first display without allowing the user at the second display to input treatment parameters or accept a treatment plan.

Clause 111. The user interface of any of the preceding clauses, wherein the treatment plan can only be accepted at the first display.

Clause 112. The user interface of any of the preceding clauses, further comprising a monitor control icon configured to receive a user input to adjust user input controls available at the first display and the second display.

Clause 113. The user interface of any of the preceding clauses, wherein the user input controls are configured to allow the user at the first display to allow the user at the second display to input annotations at the first display in a first configuration, or to disable user input at the second display in a second configuration.

Clause 114. The user interface of any of the preceding clauses, wherein the monitor control icon can only receive input from the first display.

Clause 115. A system to treat a patient, the system comprising: a processor; a touch screen display configured to provide the user interface of any of the preceding claims.

Clause 116. A method of receiving a user input, the method comprising: providing the user interface as in any of the preceding claims; and receiving the user input.

Clause 117. A method of use, the method comprising: providing a user input to the user interface of any of the preceding claims.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A user interface to operate a robotic system to perform a surgery, the user interface comprising:
instructions stored on a computer readable medium, which when executed by one or more processors, cause the one or more processors to:
provide a plurality of images on an image display area of a touchscreen display;
provide a control panel on the touchscreen display, the control panel partitioned from the image display area, the control panel comprising a plurality of features to receive a plurality of user inputs in response to the user touching the plurality of features;
display a current mode of a plurality of modes of the system.

2. The user interface of claim 1, wherein the control panel is located below the image display area.

3. The user interface of claim 1, wherein the instructions are configured to select in sequence: 1) an ultrasound image source as the primary image source for the primary image display area in a first mode or sub-mode to insert an ultrasound probe into a patient; 2) an endoscope image source as the primary image source for the primary display area and the ultrasound image as the secondary image source for the secondary image display area in a second mode or sub-mode to insert a treatment probe comprising an endoscope into the patient; and 3) the ultrasound image source as the primary image source for the primary image display area and the endoscope image source as the secondary image source for the secondary image display area for each of a plurality of subsequent modes or sub-modes.

4. The user interface of claim 3, wherein the plurality of subsequent modes or sub-modes comprises a planning mode or sub-mode and a treatment mode or sub-mode.

5. The user interface of claim 3, wherein the plurality of subsequent modes or sub-modes comprises three or more of a probe alignment mode or sub-mode, an angle and depth mode or sub-mode, a registration mode or sub-mode, a profile mode or sub-mode, or a treatment mode or sub-mode.

6. The user interface of claim 1, wherein the user interface is configured to automatically select an image source for an image to display in the image display area, the image source and the image corresponding to a current mode and a current sub-mode of the system.

7. The user interface of claim 3, wherein the instructions are configured to select a primary image source for a primary image display area and a secondary image source corresponding to a secondary image display area in response to the current mode and the current sub-mode of the system.

8. The user interface of claim 7, wherein the instructions are configured to select an ultrasound image source as the primary image source in a mode corresponding to insertion of an ultrasound probe into the patient and an endoscope image source for the primary image display area in a mode corresponding to insertion of a surgical treatment probe into the patient.

9. The user interface of claim 8, wherein the processor is configured to select the ultrasound probe as the primary image source and the endoscope image source as the secondary image source for a plurality of modes subsequent to the mode corresponding to insertion of the treatment probe into the patient.

10. The user interface of claim 9, wherein the plurality of modes subsequent to the mode of inserting the treatment probe comprises a planning mode and a treatment mode.

11. The user interface of claim 10, wherein the plurality of modes subsequent to the mode of inserting the treatment probe comprises two or more of an alignment mode, an angle and depth mode, a registration mode, a profile mode or a treatment mode.

12. The user interface of claim 10, wherein the plurality of modes subsequent to inserting the treatment probe comprises at least four modes in which the primary image source comprises the ultrasound probe and the secondary image source comprises the endoscope.

13. The user interface of claim 6, wherein the user interface is configured to automatically select one or more of an endoscope image, an ultrasound image, a transverse ultrasound image or a longitudinal image to display in the image display area.

14. The user interface of claim 6, wherein the user interface is configured to display a primary image from a first image source and a secondary image from a secondary image source in the image display area.

15. The user interface of claim 14, wherein the primary image source and the secondary image source correspond to the current mode and the current sub mode of the mode.

16. The user interface of claim 14, wherein the user interface is configured to automatically display a real time endoscope image from an endoscope in the primary image display area and a real time ultrasound image from an ultrasound device in the secondary image display area in a first mode and to automatically display the real time ultrasound image in the primary image display area and the endoscope image in the secondary image display area in a second mode.

17. The user interface of claim 16, wherein in the second mode the user interface is configured to automatically display a real time transverse ultrasound image in a first sub-mode of the second mode and to automatically display a real time longitudinal ultrasound image in a second sub-mode of the second mode.

18. The user interface of claim 17, wherein the user interface is configured to toggle between the real time transverse ultrasound image and the real time longitudinal ultrasound image in the first sub-mode of the second mode and between the real time longitudinal ultrasound image and the real time transverse image in the second sub-mode of the second mode.

19. The user interface of claim 17, wherein the real time transverse ultrasound image and the real time longitudinal ultrasound image shown in the image display area remain above the control panel when toggled.

20. The user interface of claim 1, wherein the user interface is configured to highlight a current mode of the plurality of modes.

21.-117. (canceled)
